SEQUENCE LISTING (53 sequences)

SEQ ID NO: 1 - protein, 35 aa, Homo sapiens
PVAIHADQLTPTWRVYSTGSNVFQTRAGCLIGAEH

SEQ ID NO: 2 - protein, 30 aa, Homo sapiens
LHRSYLTPGDSSSGWTAGAAAYYVGYLQPR

SEQ ID NO: 3 - protein, 18 aa, Homo sapiens
VLPFNDGVYFASTEKSNI

SEQ ID NO: 4 - protein, 39 aa, Homo sapiens
WMESEFRVYSSANNCTFEYVSQPFLMDLEGKQGNFKNLR

SEQ ID NO: 5 - protein, 18 aa, Homo sapiens
PVAIHADQLTPTWRVYST

SEQ ID NO: 6 - protein, 20 aa, Homo sapiens
PVAIHADQLTPTWRVYSTGS

SEQ ID NO: 7 - protein, 16 aa, Homo sapiens
DQLTPTWRVYSTGSNV

SEQ ID NO: 8 - protein, 14 aa, Homo sapiens
TWRVYSTGSNVFQT

SEQ ID NO: 9 - protein, 18 aa, Homo sapiens
RVYSTGSNVFQTRAGCLI

SEQ ID NO: 10 - protein, 16 aa, Homo sapiens
RVYSTGSNVFQTRAGC

SEQ ID NO: 11 - protein, 7 aa, Homo sapiens
RVYSTGS

SEQ ID NO: 12 - protein, 18 aa, Homo sapiens
VYSTGSNVFQTRAGCLIG

SEQ ID NO: 13 - protein, 14 aa, Homo sapiens
GSNVFQTRAGCLIG

SEQ ID NO: 14 - protein, 14 aa, Homo sapiens
SNVFQTRAGCLIGA

SEQ ID NO: 15 - protein, 14 aa, Homo sapiens
VFQTRAGCLIGAEH

SEQ ID NO: 16 - protein, 15 aa, Homo sapiens
GSNVFQTRAGCLIGA

SEQ ID NO: 17 - protein, 15 aa, Homo sapiens
GSNVFQTRAGCLIGA

SEQ ID NO: 18 - protein, 9 aa, Homo sapiens
FQTRAGCLI

SEQ ID NO: 19 - protein, 29 aa, Homo sapiens
LHRSYLTPGDSSSGWTAGAAAYYVGYLQP

SEQ ID NO: 20 - protein, 14 aa, Homo sapiens
LHRSYLTPGDSSSG

SEQ ID NO: 21 - protein, 16 aa, Homo sapiens
HRSYLTPGDSSSGWTA

SEQ ID NO: 22 - protein, 20 aa, Homo sapiens
SYLTPGDSSSGWTAGAAAYY

SEQ ID NO: 23 - protein, 9 aa, Homo sapiens
SYLTPGDSS

SEQ ID NO: 24 - protein, 20 aa, Homo sapiens
YLTPGDSSSGWTAGAAAYYV

SEQ ID NO: 25 - protein, 20 aa, Homo sapiens
LTPGDSSSGWTAGAAAYYVG

SEQ ID NO: 26 - protein, 16 aa, Homo sapiens
GWTAGAAAYYVGYLQP

SEQ ID NO: 27 - protein, 21 aa, Homo sapiens
DSSSGWTAGAAAYYVGYLQPR

SEQ ID NO: 28 - protein, 15 aa, Homo sapiens
DSSSGWTAGAAAYYV

SEQ ID NO: 29 - protein, 9 aa, Homo sapiens
GWTAGAAAY

SEQ ID NO: 30 - protein, 9 aa, Homo sapiens
WTAGAAAYY

SEQ ID NO: 31 - protein, 15 aa, Homo sapiens
TAGAAAYYVGYLQPR

SEQ ID NO: 32 - protein, 9 aa, Homo sapiens
TAGAAAYYV

SEQ ID NO: 33 - protein, 15 aa, Homo sapiens
VLPFNDGVYFASTEK

SEQ ID NO: 34 - protein, 14 aa, Homo sapiens
VLPFNDGVYFASTE

SEQ ID NO: 35 - protein, 8 aa, Homo sapiens
VYFASTEK

SEQ ID NO: 36 - protein, 15 aa, Homo sapiens
FNDGVYFASTEKSNI

SEQ ID NO: 37 - protein, 15 aa, Homo sapiens
FNDGVYFASTEKSNI

SEQ ID NO: 38 - protein, 9 aa, Homo sapiens
GVYFASTEK

SEQ ID NO: 39 - protein, 32 aa, Homo sapiens
VYSSANNCTFEYVSQPFLMDLEGKQGNFKNLR

SEQ ID NO: 40 - protein, 18 aa, Homo sapiens
VYSSANNCTFEYVSQPFL

SEQ ID NO: 41 - protein, 20 aa, Homo sapiens
YSSANNCTFEYVSQPFLMDL

SEQ ID NO: 42 - protein, 18 aa, Homo sapiens
YSSANNCTFEYVSQPFLM

SEQ ID NO: 43 - protein, 20 aa, Homo sapiens
CTFEYVSQPFLMDLEGKQGN

SEQ ID NO: 44 - protein, 20 aa, Homo sapiens
TFEYVSQPFLMDLEGKQGNF

SEQ ID NO: 45 - protein, 18 aa, Homo sapiens
FEYVSQPFLMDLEGKQGN

SEQ ID NO: 46 - protein, 20 aa, Homo sapiens
VSQPFLMDLEGKQGNFKNLR

SEQ ID NO: 47 - protein, 7 aa, Homo sapiens
MDLEGKQ

SEQ ID NO: 48 - protein, 24 aa, Homo sapiens
WMESEFRVYSSANNCTFEYVSQPF

SEQ ID NO: 49 - protein, 9 aa, Homo sapiens
WMESEFRVY

SEQ ID NO: 50 - protein, 15 aa, Homo sapiens
ESEFRVYSSANNCTF

SEQ ID NO: 51 - protein, 9 aa, Homo sapiens
YSSANNCTF

SEQ ID NO: 52 - protein, 15 aa, Homo sapiens
SSANNCTFEYVSQPF

SEQ ID NO: 53 - protein, 9 aa, Homo sapiens
SANNCTFEY

(19) United States
(12) Patent Application Publication
(10) Pub. No.: US 2024/0131149 A1
(43) Pub. Date: Apr. 25, 2024

(54) AN ISOLATED POLYPEPTIDE

(71) Applicant: UNIVERSITY OF ULSTER, COLERAINE, LONDONDERRY (GB)

(72) Inventor: PRIYANK SHUKLA, LONDONDERRY (GB)

(21) Appl. No.: 18/278,191

(22) PCT Filed: Feb. 24, 2022
(86) PCT No.: PCT/EP2022/054654

§ 371 Date: Aug. 22, 2023

(30) Foreign Application Priority Data
Feb. 24, 2021 (GB) 2102598.6

(51) Int. Cl. A61K 39/215; A61P 31/14; C07K 14/005

(52) U.S. Cl. CPC A61K 39/215; A61P 31/14; C07K 14/005

(57) ABSTRACT

The present invention relates to an isolated polypeptide that finds utility in generating an immune response against a virus in a subject. Also disclosed are immunogenic compositions comprising the isolated polypeptide, generating an immune response against a virus in a subject, and preventing or treating a viral infection in a subject, in particular a severe acute respiratory syndrome coronavirus 2 (SARS-CoV-2) infection.

### FIELD OF THE INVENTION

The present invention relates to an isolated polypeptide that finds utility in generating an immune response against a virus in a subject. Also disclosed are immunogenic compositions comprising the isolated polypeptide, generating an immune response against a virus in a subject, and preventing or treating a viral infection in a subject, in particular a severe acute respiratory syndrome coronavirus 2 (SARS-CoV-2) infection.

### BACKGROUND TO THE INVENTION

The rise of the pandemic of Severe Acute Respiratory Syndrome Coronavirus 2 (SARS-CoV-2) started in December 2019 with the reports of severe pneumonia cases of unknown aetiology from the city of Wuhan by the Chinese Centre for Disease Control (China CDC). Coronaviruses (CoV) are enveloped positive-stranded RNA-viruses belonging to a family of zoonotic viruses, which infect a variety of mammals including bats and humans. CoV are implicated in previous outbreaks such as Middle East Respiratory Syndrome (MERS-CoV), Severe Acute Respiratory Syndrome (SARS-CoV) and most recently SARS-CoV-2, which has created an urgent need to develop diagnostics, therapeutics and vaccines against SARS-CoV-2.

CoV spike glycoproteins promote entry into host cells by binding with angiotensin converting enzyme 2 (ACE2) receptor and are the main target of neutralizing antibodies. Similar to other CoVs, such as MERS-CoV and SARS-CoV, SARS-CoV-2 also utilises spike glycoprotein to gain entry into host cells and has been proposed earlier as a potential vaccine target in MERS-CoV and SARS-CoV. It is also suggested that the majority of SARS-CoV-2 vaccine candidates which are currently under clinical trials target the spike protein and its variants as the primary antigen.

Once viruses gain entry into host-cells, they direct the host-cell machinery to produce proteins and replicate their genome in an effort to replicate themselves. Vaccines protect us against these viruses by eliciting immune responses mediated by B- and T-cells. These cells mount effector functions when recognizing an antigenic determinant, called an epitope. B-cell epitopes are the residues in the antigen which bind to antibodies produced by B-cells. These specific bindings prevent interaction of pathogens with host cells and neutralize them. T-cell epitopes are the residues in the antigen that bind with major histocompatibility complex (MHC) class-I or class-II molecules to form a peptide-MHC complex, which is recognised by T-cell receptors and activates a T-cell response. In the case of humans, MHC class-I or class-Il molecules are also known as human leukocyte antigen (HLA). These HLA proteins are further classified based on their corresponding gene's locus in the human genome. The alleles of HLA genes dictate the binding specificity of each HLA protein to specific antigens.

Peptide-based vaccines are considered safer than traditional vaccines as the peptide-based vaccines avoid any possibility of unwanted host immune responses to any unnecessary material carried by attenuated microorganisms. Furthermore, peptide-based vaccines do not require in vitro culture of pathogens, which in some cases is difficult to perform. The core idea behind developing peptide-based vaccines is to identify the minimal immunogenic regions of a protein which can initiate both antibody and cell mediated (B-cell, T-cell or natural killer cell) immune responses. With advances in technology, developing a recombinant vaccine from selected immunogenic peptides is considered beneficial.

Although some vaccines have been rolled-out in some major developed and developing countries, a number of vaccine candidates are still under clinical trials at various laboratories across the world in non-profit, public, academic, multinational pharmaceutical companies and other industries. Considering the variable nature of the virus, which continues to mutate and evolve (https://www.cdc.gov/coronavirus/2019-ncov/transmission/variant.html), persistent and timely efforts are needed to develop better vaccine candidates. The production, storage, handling and transport of these vaccines should be cost-effective as well at the same time, so that they are useful for all the economies around the globe.

### SUMMARY OF THE INVENTION

According to a first aspect of the present invention there is provided an isolated polypeptide having the amino acid sequence defined in any one of SEQ ID NO:1, SEQ ID NO:2, SEQ ID NO:3, and SEQ ID NO:4 or an amino acid sequence having at least 40% identity thereto, or a fragment each thereof.

Optionally, the isolated polypeptide has the amino acid sequence defined in any one of SEQ ID NO:2, SEQ ID NO:4, SEQ ID NO:3, and SEQ ID NO:1 or an amino acid sequence having at least 40% identity thereto, or a fragment each thereof.

Optionally, the isolated polypeptide has the amino acid sequence defined in order of preference any one of SEQ ID NO:2, SEQ ID NO:4, SEQ ID NO:3, and SEQ ID NO:1 or an amino acid sequence having at least 40% identity thereto, or a fragment each thereof.

Preferably, the isolated polypeptide has the amino acid sequence defined in SEQ ID NO:2 or an amino acid sequence having at least 40% identity thereto, or a fragment each thereof.

Optionally, the isolated polypeptide has the amino acid sequence PVAIHADQLTPTWRVYSTGSNVFQTRAGCLIGAEH or an amino acid sequence having at least 40% identity thereto, or a fragment each thereof.

Optionally, the isolated polypeptide has the amino acid sequence LHRSYLTPGDSSSGWTAGAAAYYVGYLQPR or an amino acid sequence having at least 40% identity thereto, or a fragment each thereof.

Optionally, the isolated polypeptide has the amino acid sequence VLPFNDGVYFASTEKSNI or an amino acid sequence having at least 40% identity thereto, or a fragment each thereof.

Optionally, the isolated polypeptide has the amino acid sequence WMESEFRVYSSANNCTFEYVSQPFLMDLEGKQGNFKNLR or an amino acid sequence having at least 40% identity thereto, or a fragment each thereof.

Preferably, the isolated polypeptide has the amino acid sequence LHRSYLTPGDSSSGWTAGAAAYYVGYLQPR or an amino acid sequence having at least 40% identity thereto, or a fragment each thereof.

Optionally, the isolated polypeptide has an amino acid sequence having at least 40% identity to the amino acid sequence defined in any one of SEQ ID NO:2, SEQ ID NO:4, SEQ ID NO:3, and SEQ ID NO:1, or a fragment each thereof.

Optionally, the isolated polypeptide has an amino acid sequence having at least 40%, optionally at least 50%, optionally at least 60%, optionally at least 70%, optionally at least 80%, optionally at least 90%, optionally at least 95%, optionally at least 99% identity to the amino acid sequence defined in any one of SEQ ID NO:2, SEQ ID NO:4, SEQ ID NO:3, and SEQ ID NO:1, or a fragment each thereof.

Preferably, the isolated polypeptide has an amino acid sequence having at least 90% identity to the amino acid sequence defined in any one of SEQ ID NO:2, SEQ ID NO:4, SEQ ID NO:3, and SEQ ID NO:1, or a fragment each thereof.

Optionally, the isolated polypeptide has an amino acid sequence having at least 40% identity to the amino acid sequence defined in SEQ ID NO:2, or a fragment each thereof.

Optionally, the isolated polypeptide has an amino acid sequence having at least 50% identity to the amino acid sequence defined in any one of SEQ ID NO:4, or a fragment each thereof.

Optionally, the isolated polypeptide has an amino acid sequence having at least 70% identity to the amino acid sequence defined in any one of SEQ ID NO:3, or a fragment each thereof.

Optionally, the isolated polypeptide has an amino acid sequence having at least 80% identity to the amino acid sequence defined in any one of SEQ ID NO:1, or a fragment each thereof.

Preferably, the isolated polypeptide has an amino acid sequence having at least 40% identity to the amino acid sequence defined in SEQ ID NO:2, or a fragment each thereof.

Optionally, the fragment has an amino acid sequence having at least 40%, optionally at least 50%, optionally at least 60%, optionally at least 70%, optionally at least 80%, optionally at least 90%, optionally at least 95%, optionally at least 99% identity to the amino acid sequence defined in any of SEQ ID NO:5—SEQ ID NO:19.

Preferably, the fragment has an amino acid sequence having at least 40% identity to the amino acid sequence defined in either or both of SEQ ID NO:1 and SEQ ID NO:17.

Optionally, the fragment has an amino acid sequence having at least 40%, optionally at least 50%, optionally at least 60%, optionally at least 70%, optionally at least 80%, optionally at least 90%, optionally at least 95%, optionally at least 99% identity to the amino acid sequence defined in any of SEQ ID NO:20—SEQ ID NO:33.

Preferably, the fragment has an amino acid sequence having at least 40% identity to the amino acid sequence defined in either or both of SEQ ID NO:20 and SEQ ID NO:28.

Optionally, the fragment has an amino acid sequence having at least 40%, optionally at least 50%, optionally at least 60%, optionally at least 70%, optionally at least 80%, optionally at least 90%, optionally at least 95%, optionally at least 99% identity to the amino acid sequence defined in any of SEQ ID NO:34—SEQ ID NO:39.

Preferably, the fragment has an amino acid sequence having at least 40% identity to the amino acid sequence defined in either or both of SEQ ID NO:34 and SEQ ID NO:37.

Optionally, the fragment has an amino acid sequence having at least 40%, optionally at least 50%, optionally at least 60%, optionally at least 70%, optionally at least 80%, optionally at least 90%, optionally at least 95%, optionally at least 99% identity to the amino acid sequence defined in any of SEQ ID NO:41—SEQ ID NO:53.

Preferably, the fragment has an amino acid sequence having at least 40% identity to the amino acid sequence defined in either or both of SEQ ID NO:41 and SEQ ID NO:50.

According to a second aspect of the present invention there is provided an immunogenic composition comprising an isolated polypeptide according to the present invention.

Optionally, the immunogenic composition is a vaccine composition.

Accordingly, there is provided a vaccine composition comprising an isolated polypeptide of the present invention.

According to a third aspect of the present invention there is provided use of an isolated polypeptide according to the present invention in the manufacture of an immunogenic composition.

Optionally, the immunogenic composition is a vaccine composition.

Accordingly, there is provided use of an isolated polypeptide according to the present invention in the manufacture of a vaccine composition.

According to a fourth aspect of the present invention there is provided use of an isolated polypeptide according to the present invention in the manufacture of an immunogenic composition for generating an immune response in a subject.

Optionally, the immunogenic composition is a vaccine composition.

Accordingly, there is provided use of an isolated polypeptide according to the present invention in the manufacture of a vaccine composition for generating an immune response in a subject.

According to a fifth aspect of the present invention there is provided use of an isolated polypeptide according to the present invention in the manufacture of an immunogenic composition for vaccinating a subject.

Optionally, the immunogenic composition is a vaccine composition.

Accordingly, there is provided use of an isolated polypeptide according to the present invention in the manufacture of a vaccine composition for vaccinating a subject.

According to a sixth aspect of the present invention there is provided use of an isolated polypeptide according to the present invention in the manufacture of a medicament for preventing or treating a viral infection in a subject.

Accordingly, there is provided use of an isolated polypeptide according to the present invention in the manufacture of an immunogenic composition for preventing or treating a viral infection in a subject.

Accordingly, there is provided use of an isolated polypeptide in the manufacture of a vaccine composition for preventing or treating a viral infection in a subject.

According to a seventh aspect of the present invention there is provided an isolated polypeptide according to the present invention for use in generating an immune response in a subject.

Accordingly, there is provided an immunogenic composition of the present invention for use in generating an immune response in a subject.

Accordingly, there is provided a vaccine composition of the present invention for use in generating an immune response in a subject.

Optionally, the use comprises administering the isolated polypeptide, the immunogenic composition, or the vaccine composition to the subject.

According to an eighth aspect of the present invention there is provided an isolated polypeptide according to the present invention for use in vaccinating a subject.

Accordingly, there is provided an immunogenic composition of the present invention for use in vaccinating a subject.

Accordingly, there is provided a vaccine composition of the present invention for use in vaccinating a subject.

Optionally, the use comprises administering the isolated polypeptide, the immunogenic composition, or the vaccine composition to the subject.

According to a ninth aspect of the present invention there is provided an isolated polypeptide according to the present invention for use in preventing or treating a viral infection in a subject.

Accordingly, there is provided an immunogenic composition according to the present invention for use in preventing or treating a viral infection in a subject.

Accordingly, there is provided a vaccine composition according to the present invention for use in preventing or treating a viral infection in a subject.

Optionally, the use comprises administering the isolated polypeptide, the immunogenic composition, or the vaccine composition to the subject.

According to a tenth aspect of the present invention there is provided use of an isolated polypeptide according to the present invention in generating an immune response in a subject.

Accordingly, there is provided use of an immunogenic composition according to the present invention in generating an immune response in a subject.

Accordingly, there is provided use of a vaccine composition according to the present invention in generating an immune response in a subject.

According to an eleventh aspect of the present invention there is provided use of an isolated polypeptide according to the present invention in vaccinating a subject.

Accordingly, there is provided use of an immunogenic composition according to the present invention in vaccinating a subject.

Accordingly, there is provided use of a vaccine composition according to the present invention in vaccinating a subject.

According to a twelfth aspect of the present invention there is provided use of an isolated polypeptide according to the present invention in preventing or treating a viral infection in a subject.

Accordingly, there is provided use of an immunogenic composition according to the present invention in preventing or treating a viral infection in a subject.

Accordingly, there is provided use of a vaccine composition according to the present invention in preventing or treating a viral infection in a subject.

According to a thirteenth aspect of the present invention there is provided a method of generating an immune response in a subject, the method comprising the step of administering an isolated polypeptide according to the present invention to the subject.

Optionally, the method comprises administering the isolated polypeptide, the immunogenic composition, or the vaccine composition to the subject.

According to a fourteenth aspect of the present invention there is provided a method of vaccinating a subject, the method comprising the step of administering an isolated polypeptide according to the present invention to the subject.

Optionally, the method comprises administering the isolated polypeptide, the immunogenic composition, or the vaccine composition to the subject.

According to a fifteenth aspect of the present invention there is provided a method of preventing or treating a viral infection in a subject, the method comprising the step of administering an isolated polypeptide according to the present invention to the subject.

Optionally, the method comprises administering the isolated polypeptide, the immunogenic composition, or the vaccine composition to the subject.

Optionally, the immunogenic composition further comprises an adjuvant.

Optionally, the immunogenic composition further comprises an inorganic adjuvant.

Optionally, the immunogenic composition further comprises an aluminium salt.

Optionally, the immunogenic composition further comprises an aluminium salt selected form at least one of aluminium phosphate and aluminium hydroxide.

Optionally, the immunogenic composition further comprises an organic adjuvant.

Optionally, the immunogenic composition further comprises Freund's complete adjuvant or Freund's incomplete adjuvant.

Optionally, the immunogenic composition further comprises squalene or an emulsion, optionally an oil-in-water emulsion, of squalene.

Optionally, the immune response is an adaptive immune response.

Optionally, the immune response is a humoral immune response.

Optionally or additionally, the immune response is a cell-mediated immune response.

Optionally, the immune response is a humoral and cell-mediated immune response.

Optionally, the immune response is an immune response against a virus.

Optionally, the virus is selected from the family coronaviridae.

Optionally, the virus is selected from the genus betacoronavirus.

Optionally, the virus is selected from the subgenus sarbecovirus.

Optionally, the virus is selected from the species severe acute respiratory syndrome-related coronavirus.

Optionally, the virus is severe acute respiratory syndrome coronavirus 2.

Optionally, the subject is a subject selected from any one or more of East Asia, North-East Asia, South Asia, South-East Asia, South-West Asia, Europe, East Africa, West Africa, Central Africa, North Africa, South Africa, West Indies, North America, Central America, South America and Oceania.

Optionally, the subject is a subject selected from any one or more of East Asia, North-East Asia, South Asia, South-East Asia, South-West Asia, Europe, East Africa, West Africa, Central Africa, North Africa, South Africa, West Indies, North America, Central America, South America and Oceania; and the isolated polypeptide has the amino acid sequence defined in any one of SEQ ID NO:2, SEQ ID NO:4, SEQ ID NO:3, and SEQ ID NO:1 or an amino acid sequence having at least 40% identity thereto, or a fragment each thereof.

Optionally, the subject is a subject selected from any one or more of East Asia, North-East Asia, South Asia, South-East Asia, South-West Asia, Europe, East Africa, West Africa, Central Africa, North Africa, South Africa, West Indies, North America, Central America, South America and Oceania; and the isolated polypeptide has the amino acid sequence defined in order of preference any one of SEQ ID NO:2, SEQ ID NO:4, SEQ ID NO:3, and SEQ ID NO:1 or an amino acid sequence having at least 40% identity thereto, or a fragment each thereof.

Preferably, the subject is a subject selected from any one or more of East Asia, North-East Asia, South Asia, South-East Asia, South-West Asia, Europe, East Africa, West Africa, Central Africa, North Africa, South Africa, West Indies, North America, Central America, South America and Oceania; and the isolated polypeptide has the amino acid sequence defined in any one of SEQ ID NO:2 or an amino acid sequence having at least 40% identity thereto, or a fragment each thereof.

Optionally, the subject is a subject selected from South-East Asia and the isolated polypeptide has the amino acid sequence defined in any one of SEQ ID NO:2, SEQ ID NO:3, SEQ ID NO:4, and SEQ ID NO:1 or an amino acid sequence having at least 40% identity thereto, or a fragment each thereof.

Optionally, the subject is a subject selected from South-East Asia and the isolated polypeptide has the amino acid sequence defined in order of preference any one of SEQ ID NO:2, SEQ ID NO:3, SEQ ID NO:4, and SEQ ID NO:1 or an amino acid sequence having at least 40% identity thereto, or a fragment each thereof.

Preferably, the subject is a subject selected from South-East Asia and the isolated polypeptide has the amino acid sequence defined in any one of SEQ ID NO:2 or an amino acid sequence having at least 40% identity thereto, or a fragment each thereof.

Optionally, the subject is a subject selected from North Africa and the isolated polypeptide has the amino acid sequence defined in any one of SEQ ID NO:2, SEQ ID NO:4, SEQ ID NO:1, and SEQ ID NO:3 or an amino acid sequence having at least 40% identity thereto, or a fragment each thereof.

Optionally, the subject is a subject selected from North Africa and the isolated polypeptide has the amino acid sequence defined in order of preference any one of SEQ ID NO:2, SEQ ID NO:4, SEQ ID NO:1, and SEQ ID NO:3 or an amino acid sequence having at least 40% identity thereto, or a fragment each thereof.

Preferably, the subject is a subject selected from North Africa and the isolated polypeptide has the amino acid sequence defined in any one of SEQ ID NO:2 or an amino acid sequence having at least 40% identity thereto, or a fragment each thereof.

Optionally, the subject is a subject selected from Central America and the isolated polypeptide has the amino acid sequence defined in any one of SEQ ID NO:2, SEQ ID NO:4, SEQ ID NO:1, and SEQ ID NO:3 or an amino acid sequence having at least 40% identity thereto, or a fragment each thereof.

Optionally, the subject is a subject selected from Central America; and the isolated polypeptide has the amino acid sequence defined in order of preference any one of SEQ ID NO:2, SEQ ID NO:4, SEQ ID NO:1, and SEQ ID NO:3 or an amino acid sequence having at least 40% identity thereto, or a fragment each thereof.

Preferably, the subject is a subject selected from Central America and the isolated polypeptide has the amino acid sequence defined in any one of SEQ ID NO:2 or an amino acid sequence having at least 40% identity thereto, or a fragment each thereof.

Optionally, the isolated polypeptide according to the invention is capable of forming a complex with a major histocompatibility complex molecule.

Optionally, the method comprises forming a complex comprising (i) the isolated polypeptide according to the invention and (ii) a major histocompatibility complex molecule.

Optionally, the use comprises forming a complex comprising (i) the isolated polypeptide according to the invention and (ii) a major histocompatibility complex molecule.

Optionally, the major histocompatibility complex molecule is a major histocompatibility complex polypeptide.

Optionally, the major histocompatibility complex molecule is a class I or class II major histocompatibility complex molecule.

Optionally, the major histocompatibility complex molecule is a class I or class II major histocompatibility complex polypeptide.

Optionally, the major histocompatibility complex molecule is a major histocompatibility complex polypeptide encoded by an allele selected from one or more of HLA-A*02:06, HLA-DRB1*07:01, HLA-A*01:01, HLA-A*26:01, HLA-A*30:02, HLA-A*68:02, HLA-DPA1*01:03, HLA-DPB1*02:01, HLA-DQA1*01:01, HLA-DQA1*01:02, HLA-DQA1*04:01, HLA-DQA1*05:01, HLA-DQB1*03:01, HLA-DQB1*04:02, HLA-DQB1*05:01, HLA-DQB1*06:02, HLA-DRB1*09:01, HLA-A*03:01, HLA-A*11:01, HLA-DPA1*02:01, HLA-DPB1*05:01, HLA-DRB1*04:01, HLA-A*01:01, HLA-A*30:02, HLA-B*15:01, HLA-B*35:01, HLA-DPA1*01:03, HLA-DPB1*02:01, HLA-DRB1*04:05, HLA-DRB1*08:02, HLA-DRB1*09:01, HLA-DRB1*15:01, and HLA-DRB3*02:02.

Optionally, the major histocompatibility complex molecule is a class I major histocompatibility complex polypeptide encoded by an allele selected from one or more of HLA-A*02:06, HLA-A*01:01, HLA-A*26:01, HLA-A*30:02, HLA-A*68:02, HLA-A*03:01, HLA-A*11:01, HLA-A*01:01, HLA-A*30:02, HLA-B*15:01, and HLA-B*35:01.

Optionally or additionally, the major histocompatibility complex molecule is a class II major histocompatibility complex polypeptide encoded by an allele selected from one or more of HLA-DRB1*07:01, HLA-DPA1*01:03, HLA-DPB1*02:01, HLA-DQA1*01:01, HLA-DQA1*01:02, HLA-DQA1*04:01, HLA-DQA1*05:01, HLA-DQB1*03:01, HLA-DQB1*04:02, HLA-DQB1*05:01, HLA-DQB1*06:02, HLA-DRB1*09:01, HLA-DPA1*02:01, HLA-DPB1*05:01, HLA-DRB1*04:01, HLA-DPA1*01:03, HLA-DPB1*02:01, HLA-DRB1*04:05, HLA-DRB1*08:02, HLA-DRB1*09:01, HLA-DRB1*15:01, and HLA-DRB3*02:02.

Optionally, the complex comprises (i) an isolated polypeptide having the amino acid sequence defined in any one of SEQ ID NO:1 or an amino acid sequence having at least 40% identity thereto, or a fragment each thereof and (ii) a major histocompatibility complex molecule polypeptide encoded by an allele selected from one or more of HLA-A*02:06 and HLA-DRB1*07:01.

Optionally, the complex comprises (i) an isolated polypeptide having the amino acid sequence defined in any one of SEQ ID NO:2 or an amino acid sequence having at least 40% identity thereto, or a fragment each thereof and (ii) a major histocompatibility complex molecule polypeptide encoded by an allele selected from one or more of HLA-A*01:01, HLA-A*26:01, HLA-A*30:02, HLA-A*68:02, HLA-DPA1*01:03, HLA-DPB1*02:01, HLA-DQA1*01:01, HLA-DQA1*01:02, HLA-DQA1*04:01, HLA-DQA1*05:01, HLA-DQB1*03:01, HLA-DQB1*04:02, HLA-DQB1*05:01, HLA-DQB1*06:02, and HLA-DRB1*09:01.

Optionally, the complex comprises (i) an isolated polypeptide having the amino acid sequence defined in any one of SEQ ID NO:3 or an amino acid sequence having at least 40% identity thereto, or a fragment each thereof and (ii) a major histocompatibility complex molecule polypeptide encoded by an allele selected from one or more of HLA-A*03:01, HLA-A*11:01, HLA-DPA1*02:01, HLA-DPB1*05:01, and HLA-DRB1*04:01.

Optionally, the complex comprises (i) an isolated polypeptide having the amino acid sequence defined in any one of SEQ ID NO:4 or an amino acid sequence having at least 40% identity thereto, or a fragment each thereof and (ii) a major histocompatibility complex molecule polypeptide encoded by an allele selected from one or more of HLA-A*01:01, HLA-A*30:02, HLA-B*15:01, HLA-B*35:01, HLA-DPA1*01:03, HLA-DPB1*02:01, HLA-DRB1*04:05, HLA-DRB1*08:02, HLA-DRB1*09:01, HLA-DRB1*15:01, and HLA-DRB3*02:02.

### BRIEF DESCRIPTION OF THE DRAWINGS

Embodiments of the invention will be described with reference to the accompanying drawings in which:

FIG. 1 is a flowchart illustrating the immunoinformatics analysis pipeline, wherein oval shapes represent start/stop of the pipeline, parallelogram boxes represent input/output, rectangular boxes represent processing steps, and servers/tools/software and databases used are mentioned in parenthesis;

FIG. 2 illustrates visualisation of final four epitopes in SARS-CoV-2 spike protein (PDB ID: 6XR8), wherein regions of final four B-Cell and T-Cell consensus epitope sequences are highlighted in both A. monomer and B. trimer of the spike protein, wherein chain-A is highlighted in green, chain-B in blue and chain-C in orange, wherein Epitope 1, highlighted in yellow, is present in C-terminal domain 2 (CTD2) and epitopes 2-4, highlighted in red, magenta and cyan, respectively, are in N-terminal domain (NTD). C. Sequences of final four B-Cell and T-Cell consensus epitope, wherein RBD=Receptor Binding Domain, CTD1=C-terminal domain 1, S1/S2=S1/S2 cleavage site, CH=central helix region, CD=connector domain, HR1=heptad repeat 1, FP=fusion peptide and FPPR=fusion peptide proximal region, are indicated in FIG. 2a, wherein SARS-CoV-2 spike protein's domain information has been derived from Fig la of Cai et al., 2020 and 3D-rendering was performed using UCSF Chimera (Peterson et al., 2004);

FIG. 3 illustrates conformational stability of HLA-epitope complexes in terms of structural order parameters, i.e., root-mean square deviation (RMSD) of backbone Cα-atoms, radius of gyration (Rg) and solvent accessible surface area (SASA), wherein the left hand panel shows the structural order parameters for MHC-I molecules (HLA-A*30:02-epitope-2.2.2: green, HLA-A*01:01-epitope-2.2.3: black, HLA-A*26:01-epitope-2.2.3: red, HLA-A*30:02-epitope-2.2.3: blue and HLA-A*68:02-epitope-2.2.5: orange, and the right hand panel for the MHC-II molecules (HLA-DQA1*01:02/HLA-DQB1*06:02-epitope-2.2.1: green, HLA-DQA1*04:01/HLA-DQB1*04:02-epitope-2.2.1: blue, HLA-DQA1*05:01/HLADQB1*03:01-epitope-2.2.1: orange, HLA-DRB1*09:01-epitope-2.2.1: purple, HLA-DPA1*01:03/HLA-DPB1*02:01-epitope-2.2.4: black, HLADQA1*01:01/HLA-DQB1*05:01-epitope-2.2.4: red and HLA-DQA1*05:01/HLA-DQB1*03:01-epitope-2.2.4: light blue);

FIG. 4 illustrates molecular interactions of MHC-I-epitope complexes: A. HLA-A*30:02-epitope-2.2.2, B. HLA-A*01:01-epitope-2.2.3, C. HLA-A*26:01-epitope-2.2.3, D. HLA-A*30:02-epitope-2.2.3 and E. HLA-A*68:02-epitope-2.2.5;

FIG. 5 illustrates molecular interactions of MHC-II-epitope complexes: A. HLA-DQA1*01:02/HLA-DQB1*06:02-epitope-2.2.1, B. HLA-DQA1*04:01/HLA-DQB1*04:02-epitope-2.2.1, C. HLA-DQA1*05:01/HLA-DQB1*03:01-epitope-2.2.1, D. HLA-DRB1*09:01-epitope-2.2.1, E. HLA-DPA1*01:03/HLA-DPB1*02:01-epitope-2.2.4, F. HLA-DQA1*01:01/HLA-DQB1*05:01-epitope-2.2.4 and G. HLA-DQA1*05:01/HLA-DQB1*03:01-epitope-2.2.4;

FIG. 6 illustrates projection of the motion of the HLA molecules in phase space along the PC1 and PC2. A. HLA-A*30:02-epitope-2.2.2, B. HLA-A*01:01-epitope-2.2.3, C. HLA-A*26:01-epitope-2.2.3, D. HLA-A*30:02-epitope-2.2.3, E. HLA-A*68:02-epitope-2.2.5, F. HLA-DQA1*01:02/HLA-DQB1*06:02-epitope-2.2.1, G. HLA-DQA1*04:01/HLA-DQB1*04:02-epitope-2.2.1, H. HLA-DQA1*05:01/HLA-DQB1*03:01-epitope-2.2.1, I. HLA-DRB1*09:01-epitope-2.2.1, J. HLA-DPA1*01:03/HLA-DPB1*02:01-epitope-2.2.4, K. HLA-DQA1*01:01/HLA-DQB1*05:01-epitope-2.2.4 and L. HLA-DQA1*05:01/HLA-DQB1*03:01-epitope-2.2.4, wherein MHC-I-epitope complexes are coloured in green and MHC-II-epitope complexes in red;

FIG. 7 illustrates free energy landscape (FEL) of HLA-epitope complexes in 2D space: A. HLA-A*30:02-epitope-2.2.2, B. HLA-A*01:01-epitope-2.2.3, C. HLA-A*26:01-epitope-2.2.3, D. HLA-A*30:02-epitope-2.2.3, E. HLA-A*68:02-epitope-2.2.5, F. HLA-DQA1*01:02/HLA-DQB1*06:02-epitope-2.2.1, G. HLA-DQA1*04:01/HLA-DQB1*04:02-epitope-2.2.1, H. HLA-DQA1*05:01/HLA-DQB1*03:01-epitope-2.2.1, I. HLA-DRB1*09:01-epitope-2.2.1, J. HLA-DPA1*01:03/HLA-DPB1*02:01-epitope-2.2.4, K. HLA-DQA1*01:01/HLA-DQB1*05:01-epitope-2.2.4 and L. HLA-DQA1*05:01/HLA-DQB1*03:01-epitope-2.2.4; and

FIG. 8 illustrates free energy landscape (FEL) of HLA-epitope complexes in 3D space, wherein PC1 and PC2 are the first and second principal components of the projection of the motion of the HLA epitope complex in phase space. A. HLA-A*30:02-epitope-2.2.2, B. HLA-A*01:01-epitope-2.2.3, C. HLA-A*26:01-epitope-2.2.3, D. HLA-A*30:02-epitope-2.2.3, E. HLA-A*68:02-epitope-2.2.5, F. HLA-DQA1*01:02/HLA-DQB1*06:02-epitope-2.2.1, G. HLA-DQA1*04:01/HLA-DQB1*04:02-epitope-2.2.1, H. HLA-DQA1*05:01/HLA-DQB1*03:01-epitope-2.2.1, I. HLA-DRB1*09:01-epitope-2.2.1, J. HLA-DPA1*01:03/HLA-DPB1*02:01-epitope-2.2.4, K. HLA-DQA1*01:01/HLA-DQB1*05:01-epitope-2.2.4 and L. HLA-DQA1*05:01/HLA-DQB1*03:01-epitope-2.2.4.

### METHODS

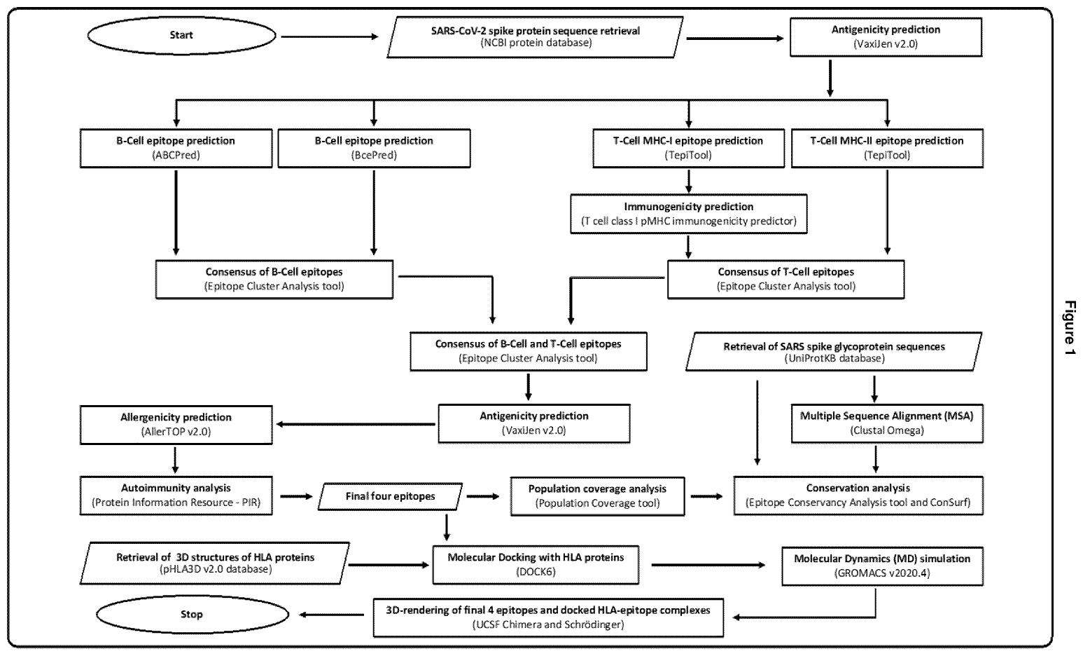

A summary flowchart of the immunoinformatics analysis pipeline is presented in FIG. 1 and each step of the pipeline is described here in detail with all the tools, software, servers, databases and specific parameters used for each of them during the analysis.

### Amino Acid Sequence Retrieval

The amino acid sequence of spike glycoprotein of SARS-CoV-2 was retrieved in FASTA format from the NCBI Protein database (NCBI Resource Coordinators. Database resources of the National Center for Biotechnology Information. Nucleic Acids Res. 2018 Jan. 4;46(D1):D8-D13. doi:10.1093/nar/gIcx1095. PMID: 29140470; PMCID: PMC5753372). GenBank ID of the protein sequence is QIC53213.1.

### Antigenicity Prediction

The VaxiJen 2.0 server (Doytchinova IA, Flower DR. VaxiJen: a server for prediction of protective antigens, tumour antigens and subunit vaccines. BMC Bioinformatics. 2007 Jan. 5;8:4. doi: 10.1186/1471-2105-8-4. PMID: 17207271; PMCID: PMC1780059) was deployed to predict antigenicity of the spike glycoprotein sequence of SARS-CoV-2. The protein sequence was entered in plain format with the following parameters; target organism: virus and threshold: 0.4.

### B-Cell Epitope Prediction

B-cell epitope prediction was performed with two different servers; i) ABCPred (Saha S, Raghava GP. Prediction of continuous B-cell epitopes in an antigen using recurrent neural network. Proteins. 2006 Oct. 1;65(1):40-8. doi: 10.1002/prot.21078. PMID: 16894596) and ii) BcePred (Saha.S and Raghava G. P. S. BcePred:Prediction of Continuous B-Cell Epitopes in Antigenic Sequences Using Physico-chemical Properties. In G.Nicosia, V.Cutello, P. J. Bentley and J.Timis (Eds.) ICARIS 2004, LNCS 3239, 197-204, Springer, 2004). ABCPred was deployed by entering the protein sequence in plain format with the following parameters; threshold: 0.8, window length: 10-20, and overlapping filter: off. BcePred was deployed by entering the protein sequence in plain format with the following parameters; physico-chemical properties: flexibility, and threshold: 1.9. The overlapping epitope sequences from the two servers were aligned by deploying Immune Epitope Database and Analysis Resource (IEDB)'s Epitope Cluster Analysis tool (Dhanda S K, Vaughan K, Schulten V, Grifoni A, Weiskopf D, Sidney J, Peters B, Sette A. Development of a novel clustering tool for linear peptide sequences. Immunology. 2018 November;155(3):331-345. doi: 10.1111/imm.12984. Epub 2018 Aug. 6. PMID: 30014462; PMCID: PMC6187223) to derive continuous stretches of consensus B-cell epitope sequences. Peptide sequences were entered in FASTA format with the following parameters; minimum sequence identity threshold: 70%, minimum peptide length: no minimum peptide length, maximum peptide length: no maximum peptide length, and clustering method: cluster-break for clear representative sequence. Further in-house filtering was applied on the clustering results by removing all singletons and those clusters which were solely based on the predicted B-cell epitope sequences of either ABCPred or BcePred servers.

### T-Cell Epitope Prediction

T-cell epitope prediction was performed for both MHC class-I and class-II response separately with IEDB's TepiTool (Paul S, Sidney J, Sette A, Peters B. TepiTool: A pipeline for computational prediction of T cell epitope candidates. Curr Protoc Immunol. 2016 Aug. 1;114:18.19.1-18.19.24. doi: 10.1002/cpim.12. PMID: 27479659; PMCID: PMC4981331). For MHC class-I epitope prediction, the protein sequence was entered in plain format along with the following parameters; host species:

human, allele class: class-I, alleles: use panel of 27 most frequent A and B alleles, peptides to be included in prediction: apply default settings for low number of peptides (i.e. only 9-mer peptides will be included and duplicate peptides will be removed), prediction method to use: IEDB recommended, and selection of predicted peptides: select peptides based on predicted percentile rank <=1. For MHC class-II epitope prediction, the protein sequence was entered in plain format along with the following parameters; host species: human, allele class: class-II, alleles: use the panel of 26 most frequent alleles, peptides to be included in prediction: apply default settings for moderate number of peptides (i.e. only 15-mer peptides will be included, number of overlapping residues fixed at 10 and duplicate peptides will be removed), prediction method to use: IEDB recommended, and selection of predicted peptides: select peptides based on predicted percentile rank <=10. The MHC class-I epitopes were further tested for immunogenicity prediction with IEDB's Class-I Immunogenicity tool (Calis J J, Maybeno M, Greenbaum J A, Weiskopf D, De Silva A D, Sette A, Keçmir C, Peters B. Properties of MHC class I presented peptides that enhance immunogenicity. PLoS Comput Biol. 2013 October;9(10):e1003266. doi: 10.1371/journal.pcbi.1003266. Epub 2013 Oct. 24. PMID: 24204222; PMCID: PMC3808449). Peptide sequences were entered in plain format along with the following parameters; specify which position to mask: custom (corresponding alleles from TepiTool's MHC class-I results were chosen from the allele dropdown menu). Epitopes with positive immunogenicity score were taken forward for building continuous stretches of consensus T-cell epitope sequences by aligning overlapping MHC-II epitope sequences using IEDB's Epitope Cluster Analysis tool (Dhanda et al., 2018) following the same parameters as described earlier. Additional in-house filtering was applied on the clustering results by removing all singletons and those clusters which were solely based on either MHC class-I or class-II epitopes.

### Selection of Final Epitopes

The overlapping B-cell and T-cell epitope sequences were aligned by deploying IEDB's Epitope Cluster Analysis tool (Dhanda et al., 2018) to derive continuous stretches of consensus B-cell and T-cell epitope sequences following the same parameters as described earlier. Further in-house filtering was applied on the clustering results by removing all singletons and those clusters which were solely based on either B-cell or T-cell epitope sequences. Consensus B-cell and T-cell epitope sequences were then tested for antigenicity using VaxiJen 2.0 server (Doytchinova et al., 2007) with the same parameters as described earlier. All the consensus epitopes which were predicted as antigenic were further scanned for allergenicity using AllerTOP 2.0 server (Dimitrov I, Bangov I, Flower DR, Doytchinova I. AllerTOP v.2—a server for in silico prediction of allergens. J Mol Model. 2014 June;20(6):2278. doi: 10.1007/s00894-014-2278-5. Epub 2014 May 31. PMID: 24878803). Peptide sequences were entered in plain format and prediction results were collated as ‘allergen’ or ‘non-allergen’. All the antigenic consensus epitopes which were predicted as allergenic were taken forward for autoimmunity check in Homo sapiens with Protein Information Resource (PIR)'s peptide search service (Wu C H, Yeh L S, Huang H, Arminski L, Castro-Alvear J, Chen Y, Hu Z, Kourtesis P, Ledley R S, Suzek B E, Vinayaka C R, Zhang J, Barker W C. The Protein Information Resource. Nucleic Acids Res. 2003 Jan. 1;31(1):345-7. doi: 10.1093/nar/gkg040. PMID: 12520019; PMCID: PMC165487). Peptide sequences were entered in plain format along with the following parameters; restrict by organism—enter an organism from UniProt: Homo sapiens [9606], options—a) UniProt/SwissProt only: yes, b) include isoforms: yes, c) UniRef100 representative sequences only: no, and d) treat leucine (L) and isoleucine (I) equivalent: no.

### Population Coverage Analysis of Selected Epitopes

All the consensus B-cell and T-cell epitope sequences which were antigenic, allergenic and passed the autoimmunity check in Homo sapiens were taken forward for population coverage analysis using IEDB's Population Coverage tool (Bui H H, Sidney J, Dinh K, Southwood S, Newman M J, Sette A. Predicting population coverage of T-cell epitope-based diagnostics and vaccines. BMC Bioinformatics. 2006 Mar. 17;7:153. doi: 10.1186/1471-2105-7-153. PMID: 16545123; PMCID: PMC1513259). This analysis was conducted for each epitope individually and then taking all the epitopes together as a set. For each epitope or epitope set, genotypic frequencies of HLA binding alleles for combined MHC class-I and class-II response were queried first against with the global population and then for each of the following continent-area-specific populations: East Asia, North-East Asia, South Asia, South-East Asia, South-West Asia, Europe, East Africa, West Africa, Central Africa, North Africa, South Africa, West Indies, North America, Central America, South America and Oceania. MHC restricted allele information for each of the epitopes was derived from the results of T-cell epitope prediction.

### Conservation Analysis of Selected Epitopes

The UniProtKB database (UniProt Consortium. UniProt: a worldwide hub of protein knowledge. Nucleic Acids Res. 2019 Jan. 8;47(D1):D506-D515. doi: 10.1093/nar/gkyl049. PMID: 30395287; PMCID: PMC6323992) was queried for the terms “spike glycoprotein” AND “sars”. This yielded a total of 18 Swiss-Prot's (Bairoch A, Apweiler R. The SWISS-PROT protein sequence data bank and its new supplement TREMBL. Nucleic Acids Res. 1996 Jan. 1;24(1):21-5. doi: 10.1093/nar/24.1.21. PMID: 8594581; PMCID: PMC145613) manually annotated and reviewed protein sequences and 232 TrEMBL's (Bairoch et al., 1996) automatically annotated and non-curated protein sequences; i.e. a total of 250 protein sequences. These sequences were manually inspected and filtered-out through in-house scripting based on the following criteria: i) sequence length lower than 1273 amino acid SARS-CoV-2 spike glycoprotein (Genbank ID: QIC53213.1), ii) annotated as ‘fragment’, iii) with any number of ‘X’ characters for un-identified residues, and iv) identical to SARS-CoV-2 spike glycoprotein sequence (Genbank ID: QIC53213.1). This led to 144 non-redundant spike glycoprotein sequences of SARS, which were taken forward for conservation analysis with two tools: i) ConSurf server (Ashkenazy H, Abadi S, Martz E, Chay O, Mayrose I, Pupko T, Ben-Tal N. ConSurf 2016: an improved methodology to estimate and visualize evolutionary conservation in macromolecules. Nucleic Acids Res. 2016 Jul. 8;44(W1):W344-50. doi: 10.1093/nar/gkw408. Epub 2016 May 10. PMID: 27166375; PMCID: PMC4987940—Berezin C, Glaser F, Rosenberg J, Paz I, Pupko T, Fariselli P, Casadio R, Ben-Tal N. ConSeq: the identification of functionally and structurally important residues in protein sequences. Bioinformatics. 2004 May 22;20(8):1322-4. doi: 10.1093/bioinformatics/bth070. Epub 2004 Feb. 10. PMID: 14871869) and ii) IEDB's Conservancy Analysis tool (Bui et al., 2006). Multiple Sequence Alignment (MSA) of these 144 non-redundant sequences was performed using EMBL-EBI's Clustal Omega tool (Madeira F, Park Y M, Lee J, Buso N, Gur T, Madhusoodanan N, Basutkar P, Tivey A R N, Potter S C, Finn R D, Lopez R. The EMBL-EBI search and sequence analysis tools APIs in 2019. Nucleic Acids Res. 2019 Jul. 2;47(W1):W636-W641. doi: 10.1093/nar/gkz268. PMID: 30976793; PMCID: PMC6602479) with default parameters. The MSA result file and phylogenetic tree files were used as input for the sequence-based ConSurf analysis. The Bayesian method was selected for the calculation of conservation scores. IEDB's Conservancy Analysis tool (Bui et al., 2006) was executed for each of the selected epitope sequence along with the 144 non-redundant spike glycoprotein sequences of SARS and following parameters; analysis type: epitope linear sequence conservancy, sequence identity threshold: 100%.

### Visualisation of Selected Epitopes and Docked HLA-Epitope Complexes

The location of selected epitope sequences in SARS-CoV-2 spike protein (PDB ID: 6XR8) (Cai Y, Zhang J, Xiao T, Peng H, Sterling SM, Walsh RM Jr, Rawson S, Rits-Volloch S, Chen B. Distinct conformational states of SARS-CoV-2 spike protein. Science. 2020 Sep 25;369(6511):1586-1592. doi: 10.1126/science.abd4251. Epub 2020 Jul 21. PMID: 32694201; PMCID: PMC7464562) was visualised using UCSF Chimera (Pettersen E F, Goddard T D, Huang C C, Couch G S, Greenblatt D M, Meng E C, Ferrin T E. UCSF Chimera—a visualization system for exploratory research and analysis. J Comput Chem. 2004 October;25(13):1605-12. doi: 10.1002/jcc.20084. PMID: 15264254) and the interaction of docked HLA-epitope complexes were visualised with the Ligand Interaction script in Maestro (Schrodinger Release 2020-4: Maestro, Schrodinger, LLC, New York, NY, 2020).

### Molecular Docking

The 3D structures of the HLA proteins (MHC-I and MHC-II) were downloaded from https://www.phla3d.com.br (Menezes Teles E Oliveira D, Melo Santos de Serpa Brandão R, Claudio Demes da Mata Sousa L, das Chagas Alves Lima F, Jamil Hadad do Monte S, Sérgio Coelho Marroquim M, Vanildo de Sousa Lima A, Gilberto Borges Coelho A, Matheus Sousa Costa J, Martins Ramos R, Socorro da Silva A. pHLA3D: An online database of predicted three-dimensional structures of HLA molecules. Hum Immunol. 2019 October;80(10):834-841. doi: 10.1016/j.humimm.2019.06.009. Epub 2019 Jun. 22. PMID: 31239187) and used as the receptors for the HLA-epitope docking. Prior to docking, the HLA receptors were processed in three steps: i) the protonation states were assigned to the protein residues using PROPKA (Søndergaard C R, Olsson M H, Rostkowski M, Jensen J H. Improved treatment of ligands and coupling effects in empirical calculation and rationalization of pKa values. J Chem Theory Comput. 2011 Jul. 12;7(7):2284-95. doi: 10.1021/ct200133y. Epub 2011 Jun. 9. PMID: 26606496) at pH of 7.4, ii) the hydrogens were added, and iii) the charges were assigned using AMBER14SB force field. The processed receptors were then subjected to AMBER energy minimization (steepest descent) for 1000 steps by putting a restraint of 100 kcal/mol Å² on all the heavy atoms to allow adjustment of the added hydrogen atoms. The initial structures of the predicted epitopes were generated using UCSF-Chimera (Pettersen et al., 2004) and energy minimization was done using the steepest descent and conjugate gradient methods to get the initial conformations of different epitopes for molecular docking. The epitopes were then docked at the antigen binding groove of the HLA molecules to predict the binding affinity of the epitopes towards the HLA molecules. Molecular docking of the predicted T-cell epitopes of SARS-CoV-2 against the HLA molecules was performed using flexible docking module of DOCK6 program with extra precision using the following parameters; max. orientations: 10000, pruning max. orientations: 10000, simplex anchor max. iterations: 1000, simplex grow max. iterations: 1000 (Allen W J, Balius T E, Mukherjee S, Brozell S R, Moustakas D T, Lang P T, Case D A, Kuntz I D, Rizzo R C. DOCK 6: Impact of new features and current docking performance. J Comput Chem. 2015 Jun. 5;36(15):1132-56. doi: 10.1002/jcc.23905. PMID: 25914306; PMCID: PMC4469538). Hundred conformations were generated for each HLA-epitope complex. Grid-based energy function was used for scoring the HLA-epitope complexes. The molecular interactions of docked complexes were analysed using the Ligand Interaction script in Maestro (Schrodinger Release 2020-4: Maestro, Schrodinger, LLC, New York, NY, 2020). The conformation with the lowest energy and favourable interactions (H-bond and hydrophobic interactions) were selected as the final complexes for further analysis.

### Molecular Dynamics (MD) Simulations

MD simulations of the HLA-epitope complexes were performed using GROMACS-2020.4 (Abraham M J, Murtola T, Schulz R, Páll S, Smith J C, Hess B, Lindahl E. GROMACS: High performance molecular simulations through multi-level parallelism from laptops to supercomputers. SoftwareX. 2015; 1-2:19-25. doi: 10.1016/j.softx.2015.06.001.—Páll S, Abraham M J, Kutzner C, Hess B, Lindahl E. Tackling exascale software challenges in molecular dynamics simulations with GROMACS. In Markidis S, Laure E (eds) Solving software challenges for exascale. EASC 2014. LNCS, Springer, Cham. 2015;8759:3-27. doi: 10.1007/978-3-319-15976-8_1) and the protein interactions were approximated using CHARMM36 force field (Huang J, MacKerell A D Jr. CHARMM36 all-atom additive protein force field: validation based on comparison to NMR data. J Comput Chem. 2013 Sep. 30;34(25):2135-45. doi: 10.1002/jcc.23354. Epub 2013 Jul. 6. PMID: 23832629; PMCID: PMC3800559). The protonation states of the HLA molecules and epitopes were determined using PROPKA (Søndergaard et al., 2011). Particle mesh Ewald summation was used to handle long-range electrostatics (Darden T, York D, Pedersen L. Particle mesh Ewald: An N.log(N) method for Ewald sums in large systems. The Journal of Chemical Physics. 1993;98(12):10089-10092. doi: 10.1063/1.464397—Essmann U, Perera L, Berkowitz M L, Darden T, Lee H, Pedersen LG. A smooth particle mesh Ewald method. The Journal of Chemical Physics. 1995;103(19):8577-8593. doi: 10.1063/1.470117) and LINCS algorithm was used to constrain hydrogen bonds (Hess B, Bekker H, Berendsen H J C, Fraaije JGEM. LINCS: A linear constraint solver for molecular simulations. Journal of Computational Chemistry. 1997;18(12):1463-1472. doi: 10.1002/(SICI)1096-987X(199709)18:12<1463::AID-JCC4>3.0.CO;2-H). Constant temperature and constant pressure were maintained using Parrinello-Danadio-Bussi thermostat (Bussi G, Donadio D, Parrinello M. Canonical sampling through velocity rescaling. J Chem Phys. 2007 Jan. 7;126(1):014101. doi: 10.1063/1.2408420. PMID: 17212484) and Parrinello-Rahman pressure (Parrinello M, Rahman A. Polymorphic transitions in single crystals: A new molecular dynamics method. Journal of Applied Physics. 1981;52(12):7182-7190. doi: 10.1063/1.328693), respectively. For MD simulations, each HLA-epitope complex was placed in the center of a cubic simulation box with a minimum padding (i.e., peptide-to-box) distance of 15 Å. The box was then solvated with TIP3P water molecules (Jorgensen W L, Chandrasekhar J, Madura J D, Impey R W, Klein M L. Comparison of simple potential functions for simulating liquid water. The Journal of Chemical Physics. 1983;79(2):926-935. doi: 10.1063/1.445869) and 0.15 M NaCl, including the neutralizing counter-ions. The periodic boundary condition was defined in x, y, and z directions, and a cut-off of 12 Å was used for the calculation of long-range interactions. The resulting systems were minimized using the steepest descent followed by conjugate gradient methods. The systems were then equilibrated for 500 ps in the NVT ensemble and subsequently for 500 ps in the NPT ensemble. The temperature and pressure were set to 310 K and 1 bar, respectively. An integration time-step of 2 fs was used. Each HLA-epitope complex was simulated for 100 ns and the snapshots were saved every 10 ps for further analysis. The obtained MD trajectories were analyzed using the tools provided in GROMACS utilities. The different structural parameters that we measured are root-mean-square deviation (RMSD), the radius of gyration (Rg), solvent-accessible-surface-area (SASA), and hydrogen bond interactions. Hydrogen bonds were defined by a distance cut-off of 3.5 Å between the donor and acceptor atom and an angle cut-off of 30°. Hydrophobic interactions were defined by the condition that the distance between two residues (i and j, with |i−j|>3) is less than 4.5 Å. Principal component analysis was performed using the projection of principal component (PCs), PC1, and PC2 along the native structure, and gmx-sham utility of GROMACS package was utilized for the free energy landscape.

### Binding Free Energy Estimation

The binding free energy of the HLA molecules (MHC-I and MHC-II) in complex with different epitopes were computed using MM-PBSA (Molecular Mechanics Poisson-Boltzmann Surface Area) (Kollman P A, Massova I, Reyes C, Kuhn B, Huo S, Chong L, Lee M, Lee T, Duan Y, Wang W, Donini O, Cieplak P, Srinivasan J, Case D A, Cheatham T E 3rd. Calculating structures and free energies of complex molecules: combining molecular mechanics and continuum models. Acc Chem Res. 2000 December;33(12):889-97. doi: 10.1021/ar000033j. PMID: 11123888.—Srinivasan J, Cheatham T E, Cieplak P, Kollman P A, Case D A. Continuum solvent studies of the stability of DNA, RNA, and phosphoramidate-DNA helices. Journal of the American Chemical Society. 1998;120(37):9401-9409. doi: 10.1021/ja981844+), which describes the protein-ligand binding affinities and stability of the protein-ligand complex. The binding free energy (ΔG_{bind}) of the protein-ligand complex, which in this case is HLA-epitope complex, can be written as:

ΔG_{bind}= [symbol] G_{complex} [symbol] − [symbol] G_{receptor} [symbol] − [symbol] G_{ligand}

[symbol]

where, G_{complex} represents the free energy of the HLA-epitope complex, G_{receptor} is the free energy of the protein (HLA molecule), G_{ligand} is the free energy of ligand (epitope) and <> represents the ensemble average. The above equation for the binding free energy can also be approximated as:

ΔG_{bind}=ΔH−TΔS≈ΔE_{MM}+ΔGₛₒₗᵥ−TΔS

where, ΔH is the change in enthalpy upon ligand binding and ΔE_{MM} is the change in the average gas-phase molecular mechanics interaction energy upon ligand binding calculated as the sum of the changes in the bonded and non-bonded interactions (electrostatics and van der Waals) upon ligand binding (ΔE_{MM}=ΔE_{bonded}+ΔE_{EEL}+ΔE_{vdW}). ΔGₛₒₗᵥ is the change in solvation free energy upon ligand binding and TΔS is the change in conformational entropy upon ligand binding at absolute temperature T. ΔGₛₒₗᵥ can be further decomposed into polar and non-polar components, i.e.,

ΔGₛₒₗᵥ=ΔG_{POL}+ΔG_{NP}

where, ΔG_{POL} and ΔG_{NP} is the change in the polar and non-polar part of the solvation free energy, respectively. In MM-PBSA approach, the polar part of solvation free energy is estimated using Poisson-Boltzmann (PB) equation and non-polar part of solvation free energy is estimated using a surface area-based approach. In this study, the binding free energy (ΔG_{bind}) of the HLA-epitope complex was computed using the MMPBSA.py script (except for the entropy term) of the AMBER Tools package (Miller B R 3rd, McGee T D Jr, Swails J M, Homeyer N, Gohlke H, Roitberg A E. MMPBSA.py: An Efficient Program for End-State Free Energy Calculations. J Chem Theory Comput. 2012 Sep. 11;8(9):3314-21. doi: 10.1021/ct300418h. Epub 2012 Aug. 16. PMID: 26605738). Single trajectory protocol was used for the MM-PBSA calculations, which only involves the simulation of the complex forms. An ionic strength of 0.15 M and a solute dielectric constant of 2 was used for the PBSA calculations. Considering the convergence issues associated with the MM-PBSA, only last 20 ns data was used for the calculations (Wang C, Nguyen P H, Pham K, Huynh D, Le T B, Wang H, Ren P, Luo R. Calculating protein-ligand binding affinities with MMPBSA: Method and error analysis. J Comput Chem. 2016 Oct. 15;37(27):2436-46. doi: 10.1002/jcc.24467. Epub 2016 Aug. 11. PMID: 27510546; PMCID: PMC5018451).

### EXAMPLES

A robust immunoinformatics pipeline (see FIG. 1) has been developed for B-cell and T-cell consensus epitope prediction for SARS-CoV-2 by meticulously choosing and deploying various tools and bioinformatics databases. The results from each step of this pipeline are disclosed in the following non-limiting examples:

### Example 1

### Antigenicity Prediction of Spike Glycoprotein of SARS-CoV-2

The amino acid sequence of the spike glycoprotein of SARS-CoV-2, which was retrieved from NCBI Protein database (Genbank ID: QIC53213.1) was 1273 amino acid residues long and was predicted to be antigenic by VaxiJen 2.0. This tool deploys an alignment-free approach based on auto cross covariance (ACC) transformation of protein sequences into uniform vectors of principal amino acid properties, and claims a prediction accuracy ranging between 70-89%. The antigenic nature of the spike glycoprotein of SARS-CoV-2 by VaxiJen 2.0 establishes it as a potential candidate to find B-cell and T-cell epitopes for designing a peptide-based vaccine.

### Example 2

### Identification of B-Cell Epitopes

In silico prediction of B-cell epitopes is done in two ways: i) sequence based (linear epitopes) and ii) conformational (3D structure) based. Linear B-cell epitope prediction uses the information about the adjacent amino acids and various methods exist for the same. However, these methods have shown to have Area Under the Curve (AUC) performance of not more than 0.6. An alternative approach is to take the consensus of different methods, which has been shown to be superior to any single predictive strategy. Therefore, it was chosen to go by the consensus from two methods: i) ABCPred, a method that applies Recurrent Neural Network (RNN), and ii) BcePred, that uses the physico-chemical properties or their combinations. The B-cell epitopes predicted by the two servers ABCPred and BcePred produced variable results. Hence a consensus of their results was developed with IEDB's Epitope Cluster Analysis tool, which yielded a total of twenty-four candidate B-cell epitope sequences (see Table 1).

TABLE 1
Twenty-four consensus sequences of B-cell epitopes predicted by ABCPred
and BcePred servers. Their length, start and end residue positions on the
Spike Glycoprotein sequence of SARS Coronavirus-2 (GenBank ID: QIC53213.1),
and protein domain in which they fall are presented here.
NTD = N-terminal domain, RBD = Receptor Binding Domain,
CTD1 = C-terminal domain 1, CTD2 = C-terminal domain 2,
S1/S2 = S1/S2 cleavage site, HR1 = heptad repeat 1,
HR2 = heptad repeat 2, CT = cytoplasmic tail and
— = not in any specific domain. SARS-COV-2 spike protein's domain
information has been derived from FIG. 1a of Cai et al., 2020.

| Epi- | | | | |
|---|---|---|---|---|
| tope | Consensus sequences of B-cell epitopes | | Residue | |
| No. | predicted by ABCPred and BcePred | Length | Positions | Domain |
| 1 | PVAIHADQLTPTWRVYSTGSNVFQTRAGCLIGAEH | 35 | 621-655 | CTD2 |
| 2 | LPVSMTKTSVDCTMYICGDSTECSNLLLQY | 30 | 727-756 | — |
| 3 | ISVTTEILPVSMTKTSVDCTMYI | 23 | 720-742 | — |
| 4 | STEKSNIIRGWIFGTTLDSKTQSLLIVNNATNVV | 34 | 94-127 | NTD |
| 5 | VYSSANNCTFEYVSQPFLMDLEGKQGNFKNLR | 32 | 159-190 | NTD |
| 6 | ATVCGPKKSTNLVKNKCVNFNFNGLTGTGVLTE | 33 | 522-554 | CTD1 |
| 7 | LHRSYLTPGDSSSGWTAGAAAYYVGYLQP | 29 | 244-272 | NTD |
| 8 | ASYQTQTNSPRRARSVASQS | 20 | 672-691 | CTD2, S1/S2, — |
| 9 | TPCSFGGVSVITPGTNTSNQVA | 22 | 588-609 | CTD1, CTD2 |
| 10 | GVYYHKNNKSWMESEFRVYSSANNCT | 26 | 142-167 | NTD |
| 11 | YNENGTITDAVDCALDPLSETKCTLKSFTVE | 31 | 279-309 | NTD, — |
| 12 | PFGEVFNATRFASVYAWNRKRISNCVA | 27 | 337-363 | RBD |
| 13 | IHVSGTNGTKRFDNPVLPFN | 20 | 68-87 | NTD |
| 14 | IAVEQDKNTQEVFAQVKQIYKTPP | 24 | 770-793 | — |
| 15 | GCVIAWNSNNLDSKVGGNYN | 20 | 431-450 | RBD |
| 16 | GNYNYLYRLFRKSNLKPF | 18 | 447-464 | RBD |
| 17 | GGFNFSQILPDPSKPSKRSFI | 21 | 798-818 | — |
| 18 | QKEIDRLNEVAKNLNESLI | 19 | 1180-1198 | HR2 |
| 19 | VLPFNDGVYFASTEK | 15 | 83-97 | NTD |
| 20 | LTGTGVLTESNKKF | 14 | 546-559 | CTD1 |
| 21 | IGKIQDSLSSTASALG | 16 | 931-946 | HR1 |
| 22 | TLVKQLSSNFGAISS | 15 | 961-975 | HR1 |
| 23 | EVRQIAPGQTGKIADY | 16 | 406-421 | RBD |
| 24 | CCSCGSCCKFDEDDSE | 16 | 1247-1262 | CT |

These epitopes ranged in size from 14-35 residues and were distributed throughout all the domains of the spike glycoprotein, suggesting that further downstream analysis was required to refine potential B-cell epitope candidates.

### Example 3

### Identification of T-Cell Epitopes

TepiTool server was deployed following the protocol previously described with IEDB's recommended method which generates consensus results of MHC-I epitopes with three prediction methods: i) ANN
an Artificial Neural Network based method also known as NetMHC (Lundegaard C, Lamberth K, Harndahl M, Buus S, Lund 0, Nielsen M. NetMHC-3.0: accurate web accessible predictions of human, mouse and monkey MHC class I affinities for peptides of length 8-11. Nucleic Acids Res. 2008 Jul. 1;36(Web Server issue):W509-12. doi: 10.1093/nar/gkn202. Epub 2008 May 7. PMID: 18463140; PMCID: PMC2447772), ii) SMM—Stabilised Matrix Method (Peters B, Sette A. Generating quantitative models describing the sequence specificity of biological processes with the stabilized matrix method. BMC Bioinformatics. 2005 May 31;6:132. doi: 10.1186/1471-2105-6-132. PMID: 15927070; PMCID: PMC1173087) and iii) CombLib (Sidney J, Assarsson E, Moore C, Ngo S, Pinilla C, Sette A, Peters B. Quantitative peptide binding motifs for 19 human and mouse MHC class I molecules derived using positional scanning combinatorial peptide libraries. Immunome Res. 2008 Jan. 25;4:2. doi: 10.1186/1745-7580-4-2. PMID: 18221540; PMCID: PMC2248166). If none of these methods were available for the sequence, the NetMHCpan method (Hoof I, Peters B, Sidney J, Pedersen LE, Sette A, Lund O, Buus S, Nielsen M. NetMHCpan, a method for MHC class I binding prediction beyond humans. Immunogenetics. 2009 Jan;61(1):1-13. doi: 10.1007/s00251-008-0341-z. Epub 2008 Nov. 12. PMID: 19002680; PMCID: PMC3319061) was used. In case of MHC-II epitopes, it generates consensus results with the following three methods: i) NN-align—an Artificial Neural Network based method (Nielsen M, Lund O. NN-align. An artificial neural network-based alignment algorithm for MHC class II peptide binding prediction. BMC Bioinformatics. 2009 Sep. 18;10:296. doi: 10.1186/1471-2105-10-296. PMID: 19765293; PMCID: PMC2753847), ii) SMM-align—a stabilising matrix alignment method (Nielsen M, Lundegaard C, Lund O. Prediction of MHC class II binding affinity using SMM-align, a novel stabilization matrix alignment method. BMC Bioinformatics. 2007 Jul. 4;8:238. doi: 10.1186/1471-2105-8-238. PMID: 17608956; PMCID: PMC1939856.) and iii) Sturniolo (Sturniolo T, Bono E, Ding J, Raddrizzani L, Tuereci O, Sahin U, Braxenthaler M, Gallazzi F, Protti M P, Sinigaglia F, Hammer J. Generation of tissue-specific and promiscuous HLA ligand databases using DNA microarrays and virtual HLA class II matrices. Nat Biotechnol. 1999 June;17(6):555-61. doi: 10.1038/9858. PMID: 10385319). If none of these methods were available for the sequence, the NetMHCllpan method (Karosiene E, Rasmussen M, Blicher T, Lund O, Buus S, Nielsen M. NetMHCllpan-3.0, a common pan-specific MHC class II prediction method including all three human MHC class II isotypes, HLA-DR, HLA-DP and HLA-DQ. Immunogenetics. 2013 October;65(10):711-24. doi: 10.1007/s00251-013-0720-y. Epub 2013 Jul. 31. PMID: 23900783; PMCID: PMC3809066) was used. A panel of the 27 most common HLA MHC-I binding A and B alleles and the 26 most common HLA MHC-II binding (DP, DQ and DR; A and B) alleles were chosen to ensure predicted epitopes cover the majority of the global population. The MHC-I epitopes length was fixed to 9-mer, as this length is suggested to be most preferable for binding majority of the ligands presented by HLA alleles. In the case of MHC-II epitopes, length was fixed to 15-mer as recommended by the TepiTool. The list of MHC-I epitopes was first refined by IEDB's Class-I Immunogenicity predictor which groups residues based on their physico-chemical properties and uses the groups as a feature for immunogenicity prediction. Following this, the consensus of MHC-I and MHC-II overlapping epitope sequences was built with IEDB's Epitope Cluster Analysis tool, which yielded a total of fifty-one candidate T-cell epitope sequences (see Table 2).

```
TABLE 2
Fifty-one consensus sequences of T-cell MHC-I and MHC-II epitopes predicted by
TepiTool server. Their length, start and end residue positions on the Spike
Glycoprotein sequence of SARS Coronavirus-2 (GenBank ID: QIC53213.1)
and protein domain in which they fall are presented here.
NTD = N-terminal domain, RBD = Receptor Binding Domain, CTD1 = C-terminal domain 1,
CTD2 = C-terminal domain 2, S2' = S2' cleavage site, FP = fusion peptide,
  FPPR = fusion peptide proximal region, HR1 = heptad repeat 1,
  CH = central helix region, HR2 = heptad repeat 2, TM = transmembrane anchor,
  CT = cytoplasmic tail and — = not in any specific domain. SARS-COV-2 spike
  protein's domain information has been derived from FIG 1a of Cai et al., 2020.
```

| Epi- | T-cell MHC-I and | | | |
|---|---|---|---|---|
| tope | MHC-II consensus | | Residue | |
| No. | epitope sequence | Length | Positions | Domain |
| 1 | NLCPFGEVFNATRFASVYAWNRKRISN | 27 | 334-360 | RBD |
| 2 | GKYEQYIKWPWYIWLGFIAGL | 21 | 1204-1224 | TM |
| 3 | FIAGLIAIVMVTIMLCC | 17 | 1220-1236 | TM, CT |
| 4 | LHSTQDLFLPFFSNVTWFHAIHV | 23 | 48-70 | NTD |
| 5 | TWFHAIHVSGTNGTK | 15 | 63-77 | NTD |
| 6 | QYTSALLAGTITSGWTFGAGAAL | 23 | 872-894 | — |
| 7 | GWTFGAGAALQIPFAMQMAY | 20 | 885-904 | — |
| 8 | SFPQSAPHGVVFLHVTYVPAQEKNF | 25 | 1051-1075 | — |
| 9 | LEPLVDLPIGINITRFQTLLALH | 23 | 223-245 | NTD |
| 10 | FVSNGTHWFVTQRNFYEP | 18 | 1095-1112 | — |
| 11 | RFDNPVLPFNDGVYF | 15 | 78-92 | NTD |
| 12 | FNDGVYFASTEKSNI | 15 | 86-100 | NTD |
| 13 | PSKPSKRSFIEDLLFNKVTLADAGFIK | 27 | 809-835 | —, S2′, FP, |
| | | | | FPPR |
| 14 | DLCFTNVYADSFVIRGDEVRQI | 22 | 389-410 | RBD |
| 15 | IRGDEVRQIAPGQTG | 15 | 402-416 | RBD |
| 16 | VTQQLIRAAEIRASANLAATKMSEC | 25 | 1008-1032 | CH |
| 17 | VVIKVCEFQFCNDPFLGVYY | 20 | 126-145 | NTD |
| 18 | WMESEFRVYSSANNCTFEYVSQPF | 24 | 152-175 | NTD |
| 19 | GLTVLPPLLTDEMIAQYTSALLAGT | 25 | 857-881 | — |
| 20 | GYQPYRVVVLSFELLHAPATVCGPK | 25 | 504-528 | RBD, CTD1 |
| 21 | LPPAYTNSFTRGVYYPDKVFRS | 22 | 24-45 | NTD |
| 22 | DSSSGWTAGAAAYYVGYLQPR | 21 | 253-273 | NTD |
| 23 | AYSNNSIAIPTNFTISVTTEILPV | 24 | 706-729 | — |
| 24 | QMAYRFNGIGVTQNVLY | 17 | 901-917 | —, HR1 |
| 25 | SRLDKVEAEVQIDRLI | 16 | 982-997 | HR1, CH |
| 26 | LKYNENGTITDAVDCALDPLSE | 22 | 277-298 | NTD |
| 27 | LREFVFKNIDGYFKIYSKHT | 20 | 189-208 | NTD |
| 28 | DVDLGDISGINASVVNIQKE | 20 | 1163-1182 | HR2 |
| 29 | TSNFRVQPTESIVRFPNITN | 20 | 315-334 | NTD, RBD |
| 30 | NGVEGFNCYFPLQSYGFQPT | 20 | 481-500 | RBD |
| 31 | LQYGSFCTQLNRALTGIAVEQDK | 23 | 754-776 | — |
| 32 | SNLKPFERDISTEIYQAGST | 20 | 459-478 | RBD |
| 33 | CDVVIGIVNNTVYDPLQPELD | 21 | 1126-1146 | — |
| 34 | AYYVGYLQPRTFLLKYNENG | 20 | 264-283 | NTD |
| 35 | STEKSNIIRGWIFGTTLDSK | 20 | 94-113 | NTD |
| 36 | ISSVLNDILSRLDKV | 15 | 973-987 | HR1, CH |
| 37 | YTMSLGAENSVAYSNNSIAIP | 21 | 695-715 | — |
| 38 | ASYQTQTNSPRRARSVASQSII | 22 | 672-693 | — |
| 39 | VGGNYNYLYRLFRKSNLKPF | 20 | 445-464 | RBD |
| 40 | DVVNQNAQALNTLVKQLSSN | 20 | 950-969 | HR1 |
| 41 | ESNKKFLPFQQFGRD | 15 | 554-568 | CTD1 |
| 42 | GSNVFQTRAGCLIGA | 15 | 639-653 | CTD2 |
| 43 | FQQFGRDIADTTDAV | 15 | 562-576 | CTD1 |
| 44 | NTQEVFAQVKQIYKT | 15 | 777-791 | — |
| 45 | DIPIGAGICASYQTQ | 15 | 663-677 | CTD2 |
| 46 | LPDDFTGCVIAWNSN | 15 | 425-439 | RBD |
| 47 | SIVRFPNITNLCPFG | 15 | 325-339 | —, RBD |
| 48 | ENQKLIANQFNSAIGK | 16 | 918-933 | HR1 |
| 49 | LQSYGFQPTNGVGYQ | 15 | 492-506 | RBD |
| 50 | PAQEKNFTTAPAICH | 15 | 1069-1083 | HR2 |
| 51 | MFVFLVLLPLVSSQC | 15 | 1-15 | —, NTD |

These epitopes ranged in size from 15-27 residues and were distributed throughout all the domains of the spike glycoprotein, again suggesting that further downstream analysis was required to refine the list of potential T-cell epitope candidates.

### Example 4

### Final B-Cell and T-Cell Consensus Epitopes

Using the IEDB's Epitope Cluster Analysis tool, eleven clusters were identified, which were based on overlapping sequences of B-cell and T-cell epitopes. The rationale behind building these consensus sequences was to ensure that the selected epitopes are capable of generating both humoral and cytotoxic immune response. We went on to further rigorously refine these eleven epitopes by conducting a battery of tests: i) antigenicity test with VaxiJen 2.0, ii) allergenicity test with AllerTOP 2.0 and iii) auto-immunity test with Protein Information Resource (PIR)'s peptide search service, which finally yielded four B-cell and T-cell consensus epitopes (see Table 3).

TABLE 3
Final four B-cell and T-cell consensus epitope sequences. Their start
and end residue positions on the Spike Glycoprotein sequence of SARS
Coronavirus-2 (GenBank ID: QIC53213.1), length and the corresponding
HLA binding alleles of T-cell epitopes are presented here.

| | | | | HLA Class-I | |
|---|---|---|---|---|---|
| | | | | binding | |
| | B-Cell and T- | | | alleles of | |
| Epi- | Cell consensus | | | constituent | HLA Class-II binding |
| tope | epitope | Residue | | T-cell | alleles of constituent |
| No. | sequence | Positions | Length | epitopes | T-cell epitopes |
| 1 | PVAIHADQLTP | 621-655 | 35 | HLA-A*02:06 | HLA-DRB1*07:01 |
| | TWRVYSTGSN | | | | |
| | VFQTRAGCLIG | | | | |
| | AEH | | | | |
| 2 | LHRSYLTPGDS | 244-273 | 30 | HLA-A*01:01, | HLA-DPA1*01:03, |
| | SSGWTAGAAA | | | HLA-A*26:01, | HLA-DPB1*02:01, |
| | YYVGYLQPR | | | HLA-A*30:02, | HLA-DQA1*01:01, |
| | | | | HLA-A*68:02 | HLA-DQA1*01:02, |
| | | | | | HLA-DQA1*04:01, |
| | | | | | HLA-DQA1*05:01, |
| | | | | | HLA-DQB1*03:01, |
| | | | | | HLA-DQB1*04:02, |
| | | | | | HLA-DQB1*05:01, |
| | | | | | HLA-DQB1*06:02, |
| | | | | | HLA-DRB1*09:01 |
| 3 | VLPFNDGVYFA | 83-100 | 18 | HLA-A*03:01, | HLA-DPA1*02:01, |
| | STEKSNI | | | HLA-A*11:01 | HLA-DPB1*05:01, |
| | | | | | HLA-DRB1*04:01 |
| 4 | WMESEFRVYS | 152-190 | 39 | HLA-A*01:01, | HLA-DPA1*01:03, |
| | SANNCTFEYVS | | | HLA-A*30:02, | HLA-DPB1*02:01, |
| | QPFLMDLEGK | | | HLA-B*15:01, | HLA-DRB1*04:05, |
| | QGNFKNLR | | | HLA-B*35:01 | HLA-DRB1*08:02, |
| | | | | | HLA-DRB1*09:01, |
| | | | | | HLA-DRB1*15:01, |
| | | | | | HLA-DRB3*02:02 |

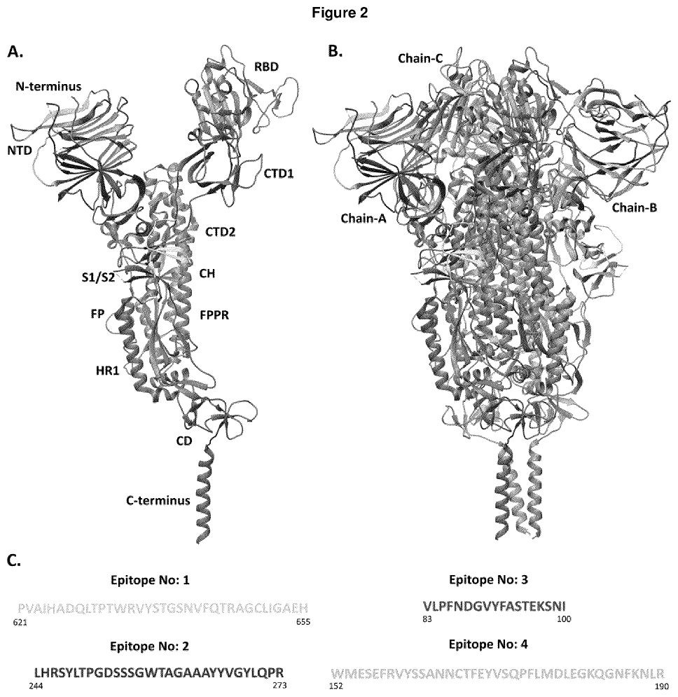

These epitopes range in size from 18-39 residues and are present in the S1 region of the SARS-CoV-2 spike glycoprotein (see FIG. 2). More specifically, epitope number 1 is present in the C-terminal domain 2 (CTD2) and epitopes numbered 2-4 are present in the N-terminal domain (NTD) (see FIG. 2).

### Example 5

### Population Coverage of Final B-Cell and T-Cell Consensus Epitopes

Global population coverage for the set of final four epitopes was computed to be 99.82% by IEDB's Population Coverage tool (see Table 4).

TABLE 4
Population coverage analysis of final four B-Cell and T-Cell consensus epitope
sequences. Values in each cell were computed by IEDB's Population Coverage
Tool and they represent the percentage of population covered by the epitope
based on the genotypic frequencies of HLA binding alleles.

| Epitope | | East | North-East | South | South-East | South-West | | East | West |
|---|---|---|---|---|---|---|---|---|---|
| No. | World | Asia | Asia | Asia | Asia | Asia | Europe | Africa | Africa |
| 1 | 19.83 | 21.66 | 14.22 | 33.97 | 12.29 | 12.60 | 23.96 | 9.22 | 9.19 |
| 2 | 98.22 | 91.24 | 97.44 | 98.18 | 92.55 | 93.43 | 99.79 | 99.94 | 99.86 |
| 3 | 65.45 | 67.63 | 77.25 | 60.62 | 72.03 | 24.33 | 55.89 | 42.45 | 75.74 |
| 4 | 88.53 | 81.45 | 86.38 | 92.55 | 47.83 | 52.60 | 98.72 | 76.45 | 76.06 |
| Set of 4 | 99.82 | 99.33 | 99.77 | 99.89 | 98.83 | 96.39 | 99.99 | 99.98 | 99.99 |

| Epitope | Central | North | South | West | North | Central | South | |
|---|---|---|---|---|---|---|---|---|
| No. | Africa | Africa | Africa | Indies | America | America | America | Oceania |
| 1 | 13.23 | 25.63 | 0.00 | 18.09 | 20.78 | 11.22 | 9.76 | 3.54 |
| 2 | 99.46 | 95.67 | 42.83 | 98.18 | 99.98 | 91.42 | 98.87 | 98.82 |
| 3 | 47.08 | 17.10 | 18.54 | 25.83 | 46.01 | NA | 48.25 | 81.74 |
| 4 | 79.90 | 65.94 | 32.48 | 43.90 | 99.71 | 33.14 | 83.73 | 87.62 |
| Set of 4 | 99.86 | 98.17 | 59.15 | 99.23 | 100.00 | 94.90 | 99.77 | 99.91 |

NA = data not available.

This computation is based on genotypic frequencies of MHC-I and MHC-II HLA binding alleles of each epitope presented in Table 3. When analysed individually, epitope number 2 in particular showed high coverage for all the continent-area-specific populations including overall world population. Epitope number 1 on the other hand had not only the least overall world population coverage of only 19.83%, but also very low coverage for each continent-area-specific populations. All four epitopes have less coverage ranging between 0.00%-59.15% in the South African population (see Table 4), which has also been previously demonstrated for other vaccines and more recently for SARS-CoV-2 vaccines. Epitope number 3 showed a high degree of variability in its population coverage ranging between 17.10%-77.25% across all the continents.

### Example 6

### Conservation of Final B-Cell and T-Cell Consensus Epitopes

With this analysis, it was sought to determine if the final four epitopes are unique for SARS-CoV-2 or can also be used against SARS-CoV; and to confirm if final four epitopes are conserved across all SARS spike glycoprotein sequences which have been isolated from different host species (viz. human, bats, civet and bovine). A high degree of protein sequence similarity has been found between SARS-CoV-2 and SARS-CoV, but not between SARS-CoV-2 and MER. Therefore, MERS spike glycoprotein sequences were excluded from this analysis. Multiple Sequence Alignment (MSA) of 144 non-redundant SARS spike glycoprotein sequences was performed with EMBL-EBI's Clustal Omega server. These sequences were annotated in UniProt database as either belonging to SARS-CoV or SARS-CoV-2 and originated from either human, bat, civet or bovine species. The MSA results were further analysed by ConSurf which graded most of the residues as variable regions, i.e. least conserved (see Table 5).

TABLE 5
Conservation analysis of final four B-Cell and T-Cell consensus epitope sequences.
Values in each cell is computed by IEDB's Conservation Analysis Tool and it
represents percentage of 114 non-redundant SARS Spike Glycoprotein sequences
retrieved from UniProtKB database which matched with epitope sequence with the
sequence similarity (percentage) described in the respective column field.
Multiple Sequence Alignment (MSA) of 114 unique SARS Spike Glycoprotein sequences
was performed with Clustal Omega and the results were analysed with ConSurf which
grades conservation of amino acids in a scale of 1-9. Amino acids in not-bold font
are variable regions (ConSurf score grade 1-3), in bold-only font are average conserved
regions (ConSurf score grade 4-6), and in underlined-bold font are highly conserved regions
(ConSurf score grade 7-9).

| Epi- | | | | | | | | | | |
|---|---|---|---|---|---|---|---|---|---|---|
| tope | B-Cell and T-Cell consensus | | | | | | | | | |
| No. | epitope sequence | 100% | 90% | 80% | 70% | 60% | 50% | 40% | 30% | 0-20% |
| 1 | PVAIHADQLTPTWRVYSTGSNVFQTRAGCLI | 1.75 | 6.14 | 77.19 | 98.25 | 98.25 | 98.25 | 98.25 | 99.12 | 100.00 |
| | GAEH | | | | | | | | | |
| 2 | LHRSYLTPGDSSSGWTAGAAAYYVGYLQPR | 1.75 | 1.75 | 1.75 | 1.75 | 1.75 | 1.75 | 1.75 | 78.95 | 100.00 |
| 3 | VLPFNDGVYFASTEKSNI | 1.75 | 1.75 | 2.63 | 87.72 | 98.25 | 99.12 | 99.12 | 99.12 | 100.00 |
| 4 | WMESEFRVYSSANNCTFEYVSQPFLMDLEG | 1.75 | 1.75 | 1.75 | 1.75 | 1.75 | 3.51 | 93.86 | 95.61 | 100.00 |
| | KQGNFKNLR | | | | | | | | | |

Interestingly, epitope number 2 which showed maximum population coverage (see Table 4), showed least conservation with IEDB's Population Coverage tool, such that 79% of 144 non-redundant sequences of SARS spike glycoprotein showed less than 30% identify with the epitope sequence (see Table 5). Epitope number 4 which was second-best in terms of population coverage (see Table 4) also showed poor conservation where 94% of 144 non-redundant sequences of SARS spike glycoprotein showed less than 40% identity with the epitope sequence (see Table 5). Epitopes numbered 1 and 3 were better conserved with 88-98% of 144 non-redundant sequences of SARS spike glycoprotein showing 70% identity with the two epitope sequences (see Table 5), but their population coverage was less than the other two epitopes (see Table 4). These results infer that the four epitopes, particularly epitopes 2 and 4 with maximum human population coverage, are unique for SARS-CoV-2 found in humans. These results also confirm that even though SARS-CoV-2 and SARS-CoV protein sequences are similar, the amino acid sequence of spike glycoprotein differ substantially when analysing different strains based on host origin, which warrants host-origin-specific vaccine development.

### Example 7

### Molecular Docking of Potential T-Cell Epitopes

Peptide-based vaccination relies on the ability of T-cells to recognize the antigens (peptide epitopes) to induce the immune response either via T-cell-dependent antibody-mediated responses or T-cell-mediated response like delayed hypersensitivity, etc. In order for a “new multi-epitope vaccine” to induce protective immunity, it should satisfy at least one of three criteria: i) the peptides must match with the epitope naturally presented to the immune cells during infection, ii) elicit an adequate immune response and iii) must have an optimal population coverage. In earlier analysis, epitope number 2 was found to satisfy all these three criteria to be considered as a potential candidate for peptide-based vaccine development against SARS-CoV-2. But, since the HLA molecules are extremely polymorphic in nature (more than 600 allelic forms, encoding diverse amino acid sequence), with the sequence diversity mostly concentrated in the peptide binding region (antigen binding groove between the two helices of MHC molecules), the binding affinity of the epitope towards different HLA molecules may differ. Therefore, to understand the binding affinity of the predicted epitope towards different HLA molecules and subsequent interaction between them, we performed molecular docking studies. The docking scores of five constituent T-cell epitopes numbered 2.2.1-2.2.5 of consensus epitope number 2 (see Table 6) towards different HLA molecules are listed in Table 7.

TABLE 6
Constituent B-cell and T-cell epitopes of the final four B-cell and
T-cell consensus epitope sequences. Their residue position,
length and HLA Class-I and Class-II binding alleles of each
constituent T-cell epitopes are presented here.

| | | | | | HLA | HLA |
|---|---|---|---|---|---|---|
| Epi- | | | | | Class-I | Class-II |
| tope | Epitope | Epitope | Residue | | binding | binding |
| no. | type | sequence | positions | Length | alleles | alleles |
| 1 | Consensus | PVAIHADQLTPTW | 621-655 | 35 | — | — |
| | of B-cell | RVYSTGSNVFQTR | | | | |
| | and T-cell | AGCLIGAEH | | | | |
| | epitopes | | | | | |
| 1.1 | Consensus | PVAIHADQLTPTW | 621-655 | 35 | — | — |
| | of B-cell | RVYSTGSNVFQTR | | | | |
| | epitopes | AGCLIGAEH | | | | |
| 1.1.1 | B-cell | PVAIHADQLTPTW | 621-638 | 18 | — | — |
| | epitope | RVYST-------- | | | | |
| | | --------- | | | | |
| 1.1.2 | B-cell | PVAIHADQLTPTW | 621-640 | 20 | — | — |
| | epitope | RVYSTGS------ | | | | |
| | | --------- | | | | |
| 1.1.3 | B-cell | ------ | 627-642 | 16 | — | — |
| | epitope | DQLTPTWRVYSTG | | | | |
| | | SNV---------- | | | | |
| | | --- | | | | |
| 1.1.4 | B-cell | --------- | 630-645 | 16 | — | — |
| | epitope | TPTWRVYSTGSNV | | | | |
| | | FQT---------- | | | | |
| 1.1.5 | B-cell | ----------- | 632-645 | 14 | — | — |
| | epitope | TWRVYSTGSNVFQ | | | | |
| | | T---------- | | | | |
| 1.1.6 | B-cell | ------------- | 634-651 | 18 | — | — |
| | epitope | RVYSTGSNVFQTR | | | | |
| | | AGCLI---- | | | | |
| 1.1.7 | B-cell | ------------- | 634-649 | 16 | — | — |
| | epitope | RVYSTGSNVFQTR | | | | |
| | | AGC------ | | | | |
| 1.1.8 | B-cell | ------------- | 634-640 | 7 | — | — |
| | epitope | RVYSTGS------ | | | | |
| | | --------- | | | | |
| 1.1.9 | B-cell | ------------- | 635-652 | 18 | — | — |
| | epitope | - | | | | |
| | | VYSTGSNVFQTRA | | | | |
| | | GCLIG--- | | | | |
| 1.1.10 | B-cell | ------------- | 639-652 | 14 | — | — |
| | epitope | ----- | | | | |
| | | GSNVFQTRAGCLI | | | | |
| | | G--- | | | | |
| 1.1.11 | B-cell | ------------- | 640-653 | 14 | — | — |
| | epitope | ------ | | | | |
| | | SNVFQTRAGCLIG | | | | |
| | | A-- | | | | |
| 1.1.12 | B-cell | ------------- | 642-655 | 14 | — | — |
| | epitope | -------- | | | | |
| | | VFQTRAGCLIGAE | | | | |
| | | H | | | | |
| 1.2 | Consensus | ------------- | 639-653 | 15 | — | — |
| | of T-cell | ----- | | | | |
| | epitopes | GSNVFQTRAGCLI | | | | |
| | | GA-- | | | | |
| 1.2.1 | T-cell | ------------- | 639-653 | 15 | — | HLA-DRB1*07:01 |
| | epitope | ----- | | | | |
| | | GSNVFQTRAGCLI | | | | |
| | | GA-- | | | | |
| 1.2.2 | T-cell | ------------- | 643-651 | 9 | HLA-A*02:06 | — |
| | epitope | --------- | | | | |
| | | FQTRAGCLI---- | | | | |
| 2 | Consensus | LHRSYLTPGDSSS | 244-273 | 30 | — | — |
| | of B-cell | GWTAGAAAYYVGY | | | | |
| | and T-cell | LQPR | | | | |
| | epitopes | | | | | |
| 2.1 | Consensus | LHRSYLTPGDSSS | 244-272 | 29 | — | — |
| | of B-cell | GWTAGAAAYYVGY | | | | |
| | epitopes | LQP- | | | | |
| 2.1.1 | B-cell | LHRSYLTPGDSSS | 244-257 | 14 | — | — |
| | epitope | G------------ | | | | |
| | | ---- | | | | |
| 2.1.2 | B-cell | - | 245-260 | 16 | — | — |
| | epitope | HRSYLTPGDSSSG | | | | |
| | | WTA---------- | | | | |
| | | --- | | | | |
| 2.1.3 | B-cell | --- | 247-266 | 20 | — | — |
| | epitope | SYLTPGDSSSGWT | | | | |
| | | AGAAAYY------ | | | | |
| | | - | | | | |
| 2.1.4 | B-cell | ---SYLTPGDSS- | 247-255 | 9 | — | — |
| | epitope | ------------- | | | | |
| | | ---- | | | | |
| 2.1.5 | B-cell | ---- | 248-267 | 20 | — | — |
| | epitope | YLTPGDSSSGWTA | | | | |
| | | GAAAYYV------ | | | | |
| 2.1.6 | B-cell | ----- | 249-268 | 20 | — | — |
| | epitope | LTPGDSSSGWTAG | | | | |
| | | AAAYYVG----- | | | | |
| 2.1.7 | B-cell | ------------- | 257-272 | 16 | — | — |
| | epitope | GWTAGAAAYYVGY | | | | |
| | | LQP- | | | | |
| 2.2 | Consensus | --------- | 253-273 | 21 | — | — |
| | of T-cell | DSSSGWTAGAAAY | | | | |
| | epitopes | YVGYLQPR | | | | |
| 2.2.1 | T-cell | --------- | 253-267 | 15 | — | HLA- |
| | epitope | DSSSGWTAGAAAY | | | | DQA1*01:02/HLA- |
| | | YV------ | | | | DQB1*06:02, |
| | | | | | | HLA- |
| | | | | | | DQA1*04:01/HLA- |
| | | | | | | DQB1*04:02, |
| | | | | | | HLA- |
| | | | | | | DQA1*05:01/HLA- |
| | | | | | | DQB1*03:01, |
| | | | | | | HLA-DRB1*09:01 |
| 2.2.2 | T-cell | ------------- | 257-265 | 9 | HLA-A*30:02 | — |
| | epitope | GWTAGAAAY---- | | | | |
| | | ---- | | | | |
| 2.2.3 | T-cell | ------------- | 258-266 | 9 | HLA- | — |
| | epitope | -WTAGAAAYY--- | | | A*01:01, | |
| | | ---- | | | HLA- | |
| | | | | | A*26:01, | |
| | | | | | HLA-A*30:02 | |
| 2.2.4 | T-cell | ------------- | 259-273 | 15 | — | HLA- |
| | epitope | -- | | | | DPA1*01:03/HLA- |
| | | TAGAAAYYVGYLQ | | | | DPB1*02:01, |
| | | PR | | | | HLA- |
| | | | | | | DQA1*01:01/HLA- |
| | | | | | | DQB1*05:01, |
| | | | | | | HLA- |
| | | | | | | DQA1*05:01/HLA- |
| | | | | | | DQB1*03:01 |
| 2.2.5 | T-cell | ------------- | 259-267 | 9 | HLA-A*68:02 | — |
| | epitope | --TAGAAAYYV-- | | | | |
| | | ---- | | | | |
| 3 | Consensus | VLPFNDGVYFAST | 83-100 | 18 | — | — |
| | of B-Cell | EKSNI | | | | |
| | and T-Cell | | | | | |
| | epitopes | | | | | |
| 3.1 | Consensus | VLPFNDGVYFAST | 83-97 | 15 | — | — |
| | of B-Cell | EK--- | | | | |
| | epitopes | | | | | |
| 3.1.1 | B-cell | VLPFNDGVYFAST | 83-96 | 14 | — | — |
| | epitope | E---- | | | | |
| 3.1.2 | B-cell | ------- | 90-97 | 8 | — | — |
| | epitope | VYFASTEK--- | | | | |
| 3.2 | Consensus | --- | 86-100 | 15 | — | — |
| | of T-cell | FNDGVYFASTEKS | | | | |
| | epitopes | NI | | | | |
| 3.2.1 | B-cell | --- | 86-100 | 15 | — | HLA- |
| | epitope | FNDGVYFASTEKS | | | | DPA1*02:01/HLA- |
| | | NI | | | | DPB1*05:01, |
| | | | | | | HLA-DRB1*04:01 |
| 3.2.2 | B-cell | ------ | 89-97 | 9 | HLA- | — |
| | epitope | GVYFASTEK--- | | | A*03:01, | |
| | | | | | HLA-A*11:01 | |
| 4 | Consensus | WMESEFRVYSSAN | 152-190 | 39 | — | — |
| | of B-Cell | NCTFEYVSQPFLM | | | | |
| | and T-Cell | DLEGKQGNFKNLR | | | | |
| | epitopes | | | | | |
| 4.1 | Consensus | ------- | 159-190 | 32 | — | — |
| | of B-cell | VYSSANNCTFEYV | | | | |
| | epitopes | SQPFLMDLEGKQG | | | | |
| | | NFKNLR | | | | |
| 4.1.1 | B-cell | ------- | 159-176 | 18 | — | — |
| | epitope | VYSSANNCTFEYV | | | | |
| | | SQPFL-------- | | | | |
| | | ------ | | | | |
| 4.1.2 | B-cell | -------- | 160-179 | 20 | — | — |
| | epitope | YSSANNCTFEYVS | | | | |
| | | QPFLMDL------ | | | | |
| | | ----- | | | | |
| 4.1.3 | B-cell | -------- | 160-177 | 18 | — | — |
| | epitope | YSSANNCTFEYVS | | | | |
| | | QPFLM-------- | | | | |
| | | ----- | | | | |
| 4.1.4 | B-cell | ------------- | 166-185 | 20 | — | — |
| | epitope | - | | | | |
| | | CTFEYVSQPFLMD | | | | |
| | | LEGKQGN----- | | | | |
| 4.1.5 | B-cell | ------------- | 167-186 | 20 | — | — |
| | epitope | -- | | | | |
| | | TFEYVSQPFLMDL | | | | |
| | | EGKQGNF---- | | | | |
| 4.1.6 | B-cell | ------------- | 168-185 | 18 | — | — |
| | epitope | --- | | | | |
| | | FEYVSQPFLMDLE | | | | |
| | | GKQGN----- | | | | |
| 4.1.7 | B-cell | ------------- | 171-190 | 20 | — | — |
| | epitope | ------ | | | | |
| | | VSQPFLMDLEGKQ | | | | |
| | | GNFKNLR | | | | |
| 4.1.8 | B-cell | ------------- | 177-183 | 7 | — | — |
| | epitope | ------------ | | | | |
| | | MDLEGKQ------ | | | | |
| | | - | | | | |
| 4.2 | Consensus | WMESEFRVYSSAN | 152-175 | 24 | — | — |
| | of T-cell | NCTFEYVSQPF-- | | | | |
| | epitopes | ------------- | | | | |
| 4.2.1 | T-cell | WMESEFRVY---- | 152-160 | 9 | HLA- | — |
| | epitope | ------------- | | | A*01:01, | |
| | | ------------- | | | HLA-B*15:01 | |
| 4.2.2 | T-cell | -- | 154-168 | 15 | — | HLA-DRB1*04:05, |
| | epitope | ESEFRVYSSANNC | | | | HLA-DRB1*08:02, |
| | | TF----------- | | | | HLA-DRB1*09:01, |
| | | ----------- | | | | HLA-DRB1*15:01, |
| | | | | | | HLA-DRB3*02:02 |
| 4.2.3 | T-cell | -------- | 160-168 | 9 | HLA-A*01:01 | — |
| | epitope | YSSANNCTF---- | | | | |
| | | ------------- | | | | |
| | | ----- | | | | |
| 4.2.4 | T-cell | --------- | 161-175 | 15 | — | HLA- |
| | epitope | SSANNCTFEYVSQ | | | | DPA1*01:03/HLA- |
| | | PF----------- | | | | DPB1*02:01 |
| | | ---- | | | | |
| 4.2.5 | T-cell | ---------- | 162-170 | 9 | HLA- | — |
| | epitope | SANNCTFEY---- | | | A*30:02, | |
| | | ------------- | | | HLA-B*35:01 | |
| | | --- | | | | |

TABLE 7
The estimated binding free energy of HLA-epitope complexes after
performing Molecular Docking with DOCK6. Grid_{VDW} and ΔE_{EEL} are
the interaction energies due to electrostatics and van der Waals
interactions, and Dock Score is the binding free energy.

| | | | | |
|---|---|---|---|---|
| | | Grid_{vdw} | Grid_{EEL} | Dock Score |
| Epitope No. | HLA alleles | (kcal/mol) | (kcal/mol) | (kcal/mol) |

MHC-I

| | | | | |
|---|---|---|---|---|
| 2.2.2 | HLA-A*30:02 | −77.11 | −20.55 | −97.67 |
| 2.2.3 | HLA-A*01:01 | −85.50 | −9.43 | −94.93 |
| | HLA-A*26:01 | −69.43 | −21.54 | −90.97 |
| | HLA-A*30:02 | −73.88 | −10.43 | −84.30 |
| 2.2.5 | HLA-A*68:02 | −77.53 | −6.50 | −84.02 |

MHC-II

| | | | | |
|---|---|---|---|---|
| 2.2.1 | HLA-DQA1*01:02/ | −78.47 | 0.62 | −77.86 |
| | HLA-DQB1*06:02 | | | |
| | HLA-DQA1*04:01/ | −104.62 | −12.89 | −117.51 |
| | HLA-DQB1*04:02 | | | |
| | HLA-DQA1*05:01/ | −104.93 | −17.13 | −122.06 |
| | HLA-DQB1*03:01 | | | |
| | HLA-DRB1*09:01 | −95.74 | −12.88 | −108.62 |
| 2.2.4 | HLA-DPA1*01:03/ | −114.10 | −22.26 | −136.36 |
| | HLA-DPB1*02:01 | | | |
| | HLA-DQA1*01:01/ | −82.91 | −0.63 | −83.54 |
| | HLA-DQB1*05:01 | | | |
| | HLA-DQA1*05:01/ | −102.13 | −10.39 | −112.52 |
| | HLA-DQB1*03:01 | | | |

The docking results indicate that these five constituent T-cell epitopes possess good binding affinity towards different HLA molecules which is in agreement with earlier results. The binding affinity for the MHC-I molecules lies in the range of −97.67 kcal/mol to −84.02 kcal/mol, while for MHC-II molecules it is in the range of −136.36 kcal/mol to −77.86 kcal/mol. In both cases, the major contributions are coming from the van der Waals interactions as hydrophobic residues dominate in the binding pocket as well as in epitopes (see Table 7). The best binding affinity for MHC-I molecules was observed for epitope number 2.2.2 against HLA-A*30:02 (-97.67 kcal/mol) and epitope number 2.2.3 against HLAA*01:01 (-94.93 kcal/mol) and HLA-A*26:01 (-90.97 kcal/mol). And, for the MHC-II molecules, epitope number 2.2.4 and 2.2.1 showed a promising binding affinity towards HLA-DPA1*01:03/HLA-DPB1*02:01, HLA-DQA1*05:01/HLA-DQB1*03:01 and HLADQA1*04:01/HLA-DQB1*04:02, respectively. It is also to be noted here that the two epitopes, i.e., 2.2.2 and 2.2.3 have 8 residues in common and essentially differ by a single residue window shift leading to a difference of one residue flanking on each side (see Table 6), but they possess a significantly different affinity towards HLAA* 30:02 (dock score differs by ˜10 kcal/mol). Quite similar is the case for epitope number 2.2.1 and 2.2.4 against HLA-DQA1*05:01/HLA-DQB1*03:01 (MHC-II). These results clearly indicate that few residues flanking the common motif in a novel designed peptide can significantly influence the overall binding preference of a peptide. Interestingly, we also observed that the same epitope can have a very different binding affinity towards different HLA molecules. For example, epitope number 2.2.4 possesses a very good binding affinity towards HLA-DPA1*01:03/HLA-DPB1*02:01, while the affinity differs by ˜52 kcal/mol for HLA-DQA1*01:01/HLA-DQB1*05:01 (see Table 7). This is expected as the binding pockets of the HLA molecules are highly diverse with respect to the amino acid sequence, and therefore the antigen binding groove has a preference towards certain amino acids to assure a stable interaction between the MHC molecule and the peptide. Overall, the results indicate that the five constituent T-cell epitopes of consensus epitope number 2 effectively bind to different HLA molecules, which is consistent with earlier results. It is also note the preference of MHC molecules towards certain peptides and vice-versa.

### Example 8

### Conformational Stability of HLA-Epitope Complexes

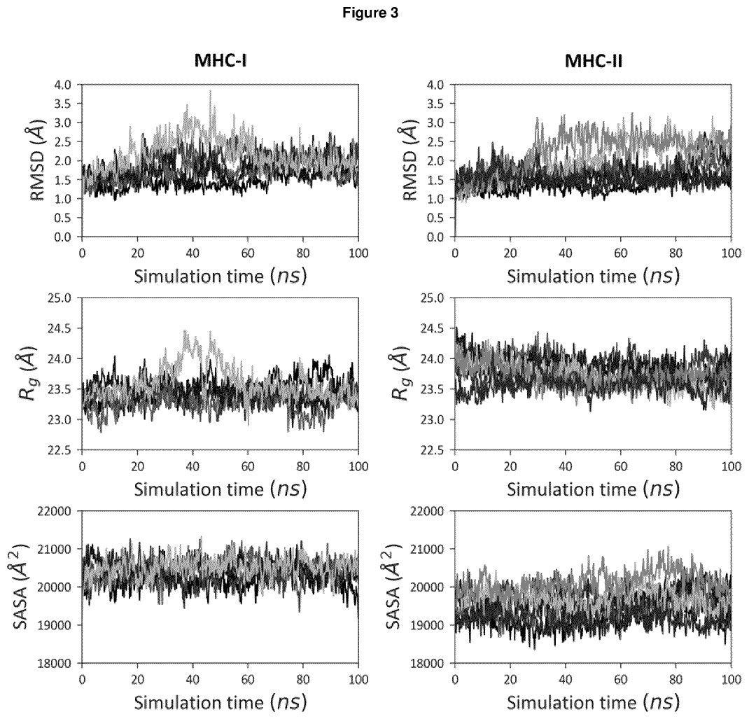

In earlier analysis, i.e., molecular docking, it has been shown that the five constituent T-cell epitopes of consensus epitope number 2 have good binding affinity towards HLA molecules, but given the approximations made in molecular docking like the “target receptor” is considered rigid, absence of water molecules, etc., it doesn't guarantee the correct binding mode for a ligand and therefore, to further confirm the results of molecular docking, binding free energy calculations were performed using MM-PBSA in combination with molecular dynamics simulation. First, the conformational stability of the HLA-epitope complexes was assessed in terms of three structural order parameters; i) root-mean square deviation (RMSD), ii) radius of gyration (Rg) and iii) solvent-accessible surface area (SASA), and the results are shown in FIG. 3.

On comparing the Cα RMSD of the HLA proteins, it can be clearly seen that all the systems attained equilibrium in the first 40 ns and remained stable thereafter, except for HLA-A*68:02-epitope-2.2.5 (orange line in the top-left panel of FIG. 3) and HLA-DQA1*05:01/HLA-DQB1*03:01-epitope-2.2.1 (orange line in the top-right panel of FIG. 3) complexes. The RMSD plot of HLA-A*68:02-epitope-2.2.5 showed slightly larger deviations in the initial 60 ns. The RMSD rose up to 3.7 Å around 43 ns, but thereafter a gradual drop is seen until 65 ns, after which a stable trajectory is seen till 100 ns. Similar behavior was observed for the HLA-DQA1*05:01/HLADQB1*03:01-epitope-2.2.1 complex. The RMSD of the HLA-DQA1*05:01/HLA-DQB1*03:01-epitope-2.2.4 is slightly higher than the other HLA-epitope complexes and fluctuates around an average value of ˜2.5 Å (blue line in the top-right panel of FIG. 3). The initial fluctuation in the RMSD indicates the spatial adjustment of the epitope in the binding site of the HLA molecules. Overall, the results suggest that all the systems achieved a steady equilibrium after 65 ns, suggesting an equilibrated and stabilized HLA-epitope interaction. To further understand the stability of the HLA-epitope complexes, we computed the radius of gyration (Rg) and solvent-accessible surface area (SASA) of the HLA proteins, as a measure of the compactness of the protein structure upon epitope binding. Except for HLA-A*68:02-epitope-2.2.5 (orange line in the center-left panel of FIG. 3) which showed major fluctuations in the initial 60 ns (Rg values ranges between 23.3 Å-24.5 Å), all the complexes showed fairly stable Rg values since the beginning of the simulations till 100 ns. Interestingly, we observed that the SASA values for all the HLA-epitope complexes remained stable throughout the length of the simulation time. All these results indicate a stable conformational dynamics of the HLA-epitope interaction and substantiate previous results.

### Example 9

### MM-PBSA Re-Scoring and Molecular Interactions Stabilizing HLA-Epitope Complexes

To further confirm the results of molecular docking and understand the molecular interactions stabilizing the HLA-epitope complex, binding free energy calculation using MM-PBSA was performed wherein both receptor flexibility as well as effect of water molecules are taken care of, which is usually ignored in molecular docking. The calculated binding free energies for the HLA-epitope complexes averaged over the snapshots extracted from the last 20 ns MD trajectories are listed in Table 8.

TABLE 8
The estimated binding free energy of HLA-epitope complexes using Molecular
Mechanics Poisson-Boltzmann Surface Area (MM-PBSA) after performing
Molecular Dynamics (MD) simulations. ΔE_{VDW} and ΔE_{EEL} are the changes in
interaction energy due to electrostatics and van der Waals interactions,
ΔG_{POL} and ΔG_{NP} are the changes in the polar and non-polar part of the
solvation free energy, and ΔG_{bind} is the change in the binding free energy.

| | | | | | | |
|---|---|---|---|---|---|---|
| Epitope | | ΔE_{VDW} | ΔE_{EEL} | ΔG_{POL} | ΔG_{NP} | ΔG_{bind} |
| No. | HLA alleles | (kcal/mol) | (kcal/mol) | (kcal/mol) | (kcal/mol) | (kcal/mol) |

MHC-I

| | | | | | | |
|---|---|---|---|---|---|---|
| 2.2.2 | HLA-A*30:02 | −65.34 ± | −125.39 ± | 141.01 ± | −7.85 ± | −57.56 ± |
| | | 5.29 | 18.93 | 17.72 | 0.4 | 4.22 |
| 2.2.3 | HLA-A*01:01 | −45.8 ± | −57.52 ± | 73.34 ± | −6.53 ± | −36.51 ± |
| | | 5.07 | 20.79 | 18.80 | 0.37 | 4.57 |
| | HLA-A*26:01 | −43.82 ± | −50.71 ± | 56.43 ± | −6.29 ± | −44.39 ± |
| | | 6.69 | 15.20 | 14.50 | 0.81 | 6.47 |
| | HLA-A*30:02 | −80.38 ± | −138.93 ± | 154.10 ± | −9.58 ± | −74.80 ± |
| | | 6.21 | 10.81 | 9.18 | 0.21 | 5.02 |
| 2.2.5 | HLA-A*68:02 | −62.65 ± | −64.73 ± | 83.47 ± | −7.48 ± | −51.39 ± |
| | | 4.22 | 17.62 | 14.83 | 0.24 | 4.36 |

MHC-II

| | | | | | | |
|---|---|---|---|---|---|---|
| 2.2.1 | HLA- | −80.76 ± | −21.02 ± | 42.68 ± | −9.19 ± | −68.28 ± |
| | DQA1*01:02/HLA- | 5.15 | 18.03 | 17.13 | 0.47 | 5.05 |
| | DQB1*06:02 | | | | | |
| | HLA- | −95.32 ± | −4.07 ± | 29.75 ± | −11.08 ± | −80.72 ± |
| | DQA1*04:01/HLA- | 7.53 | 16.18 | 17.70 | 0.94 | 6.05 |
| | DQB1*04:02 | | | | | |
| | HLA- | −66.17 ± | −11.64 ± | 32.88 ± | −8.22 ± | −53.16 ± |
| | DQA1*05:01/HLA- | 6.14 | 14.42 | 13.91 | 0.76 | 5.35 |
| | DQB1*03:01 | | | | | |
| | HLA-DRB1*09:01 | −90.06 ± | 3.23 ± | 15.09 ± | −10.08 ± | −81.81 ± |
| | | 4.98 | 9.13 | 9.47 | 0.34 | 6.06 |
| 2.2.4 | HLA- | −103.46 ± | −158.79 ± | 181.21 ± | −11.38 ± | −92.41 ± |
| | DPA1*01:03/HLA- | 4.84 | 18.29 | 17.09 | 0.39 | 5.37 |
| | DPB1*02:01 | | | | | |
| | HLA- | −68.90 ± | −53.92 ± | 71.80 ± | −8.50 ± | −59.52 ± |
| | DQA1*01:01/HLA- | 6.39 | 17.25 | 16.31 | 0.56 | 5.53 |
| | DQB1*05:01 | | | | | |
| | HLA- | −61.47 ± | −91.13 ± | 102.30 ± | −8.49 ± | −58.78 ± |
| | DQA1*05:01/HLA- | 5.17 | 14.50 | 13.98 | 0.67 | 3.86 |
| | DQB1*03:01 | | | | | |

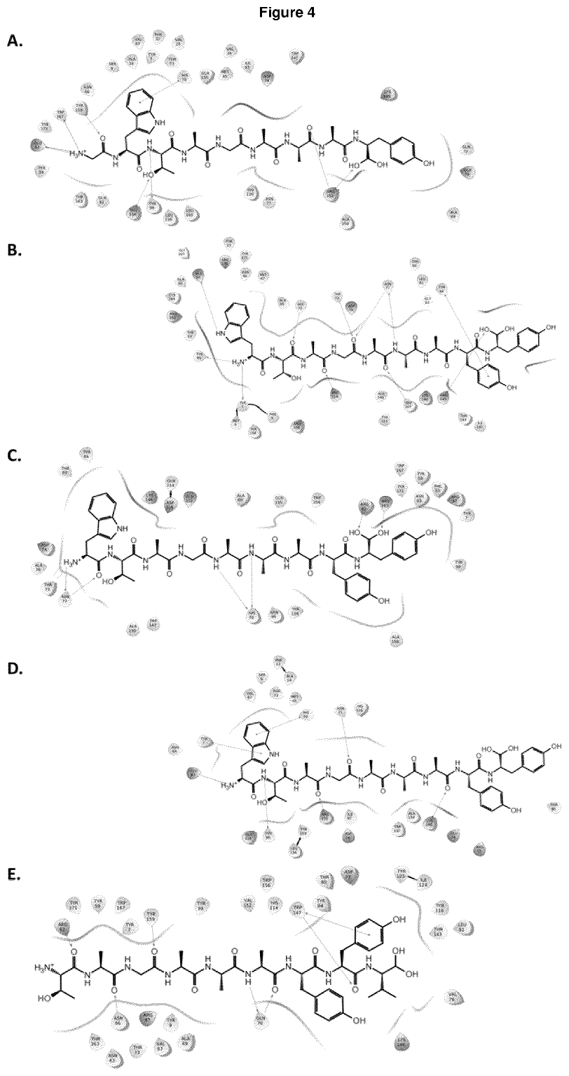

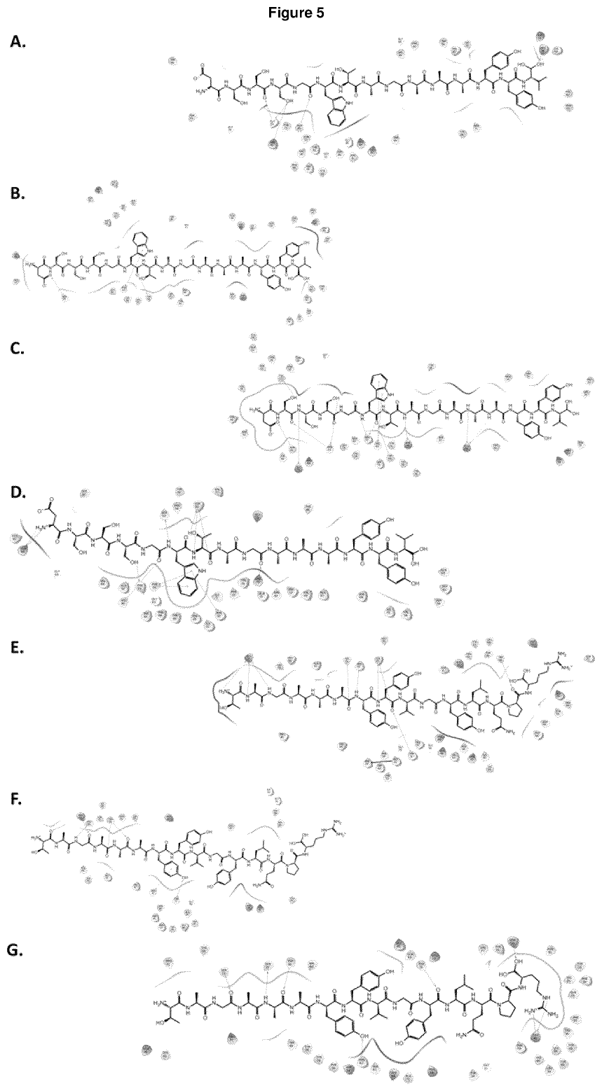

It is noted that the exact order of the ranking of binding affinities of the epitopes towards both MHC-I and MHC-II protein molecules have changed, but consistent with the results of molecular docking, all the epitopes bind favorably to the HLA molecules. The binding free energy ranges from −74.80±5.02 kcal/mol (HLA-A*30:02-epitope-2.2.3) to −36.51±4.57 kcal/mol (HLA-A*01:01-epitope-2.2.3) for MHC-I molecules and −92.41±5.37 kcal/mol (HLA-DPA1*01:03/HLA-DPB1*02:01-epitope-2.2.4) to −53.16±5.35 kcal/mol (HLA-DQA1*05:01/HLA-DQB1*03:01-epitope-2.2.1) for MHC-II molecules. Consistent with molecular docking results, the best binding affinity was observed for epitope number 2.2.4 against HLA-DPA1*01:03/HLA-DPB1*02:01 (−92.41±5.37 kcal/mol) for MHC-II molecules; however, the results changed for epitope number 2.2.1 against HLA-DQA1*01:02/HLA-DQB1*06:02 and HLA-DQA1*05:01/HLA-DQB1*03:01. The epitope number 2.2.1 now possesses the least binding affinity towards HLA-DQA1*05:01/HLA-DQB1*03:01 not HLA-DQA1*01:02/HLA-DQB1*06:02. The results also changed for the three constituent T-cell epitopes of consensus epitope number 2 against class I MHC molecules. In addition to this, we also observed a disparity between the contribution of the gas phase interactions, i.e., both van der Waals interaction as well as the electrostatic interactions between molecular docking and MM-PBSA results. Both the components now equally contribute to the stability of the MHC-I-epitope complexes, except there are cases, where electrostatic component overpowers the van der Waals interactions, as is the case with HLA-A*30:02-epitope-2.2.2 and HLA-A*30:02-epitope-2.2.3 complexes (see Table 8). In the case of MHC-II-epitope complexes, van der Waals interaction dominates in the majority of the cases with a few exceptions (see Table 8). The increase in the contribution of the electrostatic component points towards a spatial adjustment that favours the polar interactions indicating formation of mainchain-mainchain, mainchain-sidechain and sidechain-sidechain polar interactions (see Tables 7 and 8, and FIGS. 4 and 5).

The other factor that determines the binding affinity is the solvation free energy (ΔG_{POL}+Δ_{GNP}), which is neglected in molecular docking, but was found to be always positive for the HLA-epitope complexes in MM-PBSA calculations. The polar part of the solvation free energy has opposing effects much larger in magnitude than the non-polar part of solvation free energy suggesting that the solvation free energy opposes the formation of the HLA-epitope complex. But, since in all the cases, the gas phase interaction energy (ΔE_{VDW}+ΔE_{EEL}) combats the opposing effects of solvation free energy, stable HLA-epitope complexes are observed. While some discrepancies between the molecular docking results and MMPBSA calculations were observed, overall the results indicate that the epitopes possess good binding affinity towards HLA molecules, usually overwhelmed by van der Waals interaction in case of MHC-II molecules, while both van der Waals and electrostatic interactions contribute fairly to the stability of MHC-I-epitope complexes. The molecular interactions stabilizing the HLA-epitope complexes are shown in FIGS. 4 and 5, which also indicates towards the van der Waals interaction to be one of the major stabilizing forces. In addition, the average number of hydrogen bonds formed between the HLA and epitope molecules were calculated as hydrogen bonds play a critical role in stabilizing the protein-ligand interaction. The values are listed in Table 9.

TABLE 9
Number of hydrogen bonds formed between HLA-epitope complexes.
Values are averaged over last 20 ns simulation results.

| | | |
|---|---|---|
| Epitope No. | HLA alleles | No. of H-bonds |

MHC-I

| | | |
|---|---|---|
| 2.2.2 | HLA-A*30:02 | 7 ± 1 |
| 2.2.3 | HLA-A*01:01 | 3 ± 1 |
| | HLA-A*26:01 | 5 ± 1 |
| | HLA-A*30:02 | 9 ± 2 |
| 2.2.5 | HLA-A*68:02 | 5 ± 1 |

MHC-II

| | | |
|---|---|---|
| 2.2.1 | HLA-DQA1*01:02/ | 7 ± 1 |
| | HLA-DQB1*06:02 | |
| | HLA-DQA1*04:01/ | 10 ± 2 |
| | HLA-DQB1*04:02 | |
| | HLA-DQA1*05:01/ | 9 ± 1 |
| | HLA-DQB1*03:01 | |
| | HLA-DRB1*09:01 | 11 ± 2 |
| 2.2.4 | HLA-DPA1*01:03/ | 7 ± 2 |
| | HLA-DPB1*02:01 | |
| | HLA-DQA1*01:01/ | 5 ± 1 |
| | HLA-DQB1*05:01 | |
| | HLA-DQA1*05:01/ | 5 ± 1 |
| | HLA-DQB1*03:01 | |

The average number of hydrogen bonds formed between the HLA molecule's antigen presenting pocket and epitopes ranges from 3±1 (HLA-A*01:01-epitope-2.2.3) to 9±2 (HLA-A*30:02-epitope-2.2.3) for MHC-I molecules and from 5±1 (HLA-DQA1*05:01/HLA-DQB1*03:01-epitope-2.2.4) to 11±2 (HLA-DRB1*09:01-epitope-2.2.1) for MHC-II molecules, which also indicate towards the stability of the epitopes in the antigen presenting groove of the MHC molecules.

### Example 10

### Structural Motions and Conformational Redistribution of HLA-Epitope Complexes

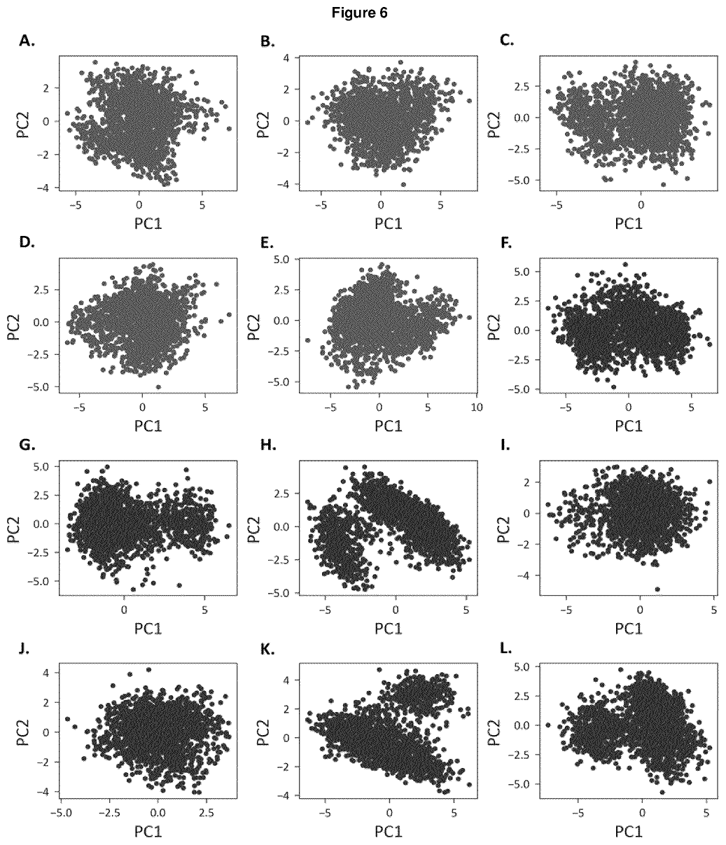

To further examine the dominant motions and conformational sampling of the HLA protein molecules upon binding of the epitopes, principal component analysis (PCA) was performed. It is generally assumed that the first ten principal components account for more than 90% motion of the protein responsible for their function. Therefore, the first ten principal components were calculated, and the conformational sampling of the HLA-epitope complexes in the essential subspace illustrating the global motions along PC1 and PC2 are shown in FIG. 6.

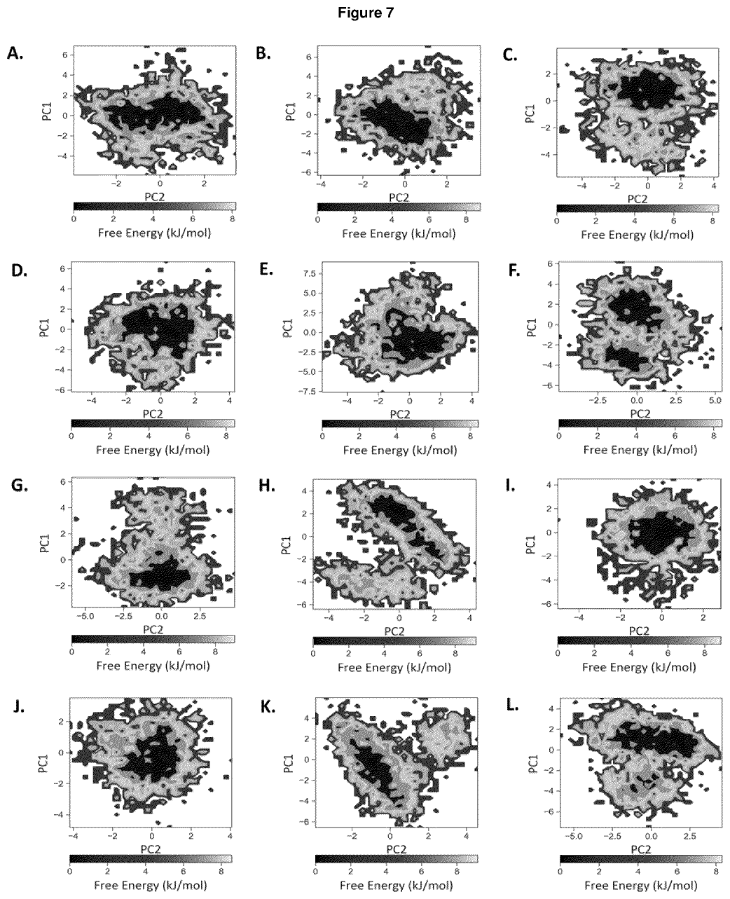

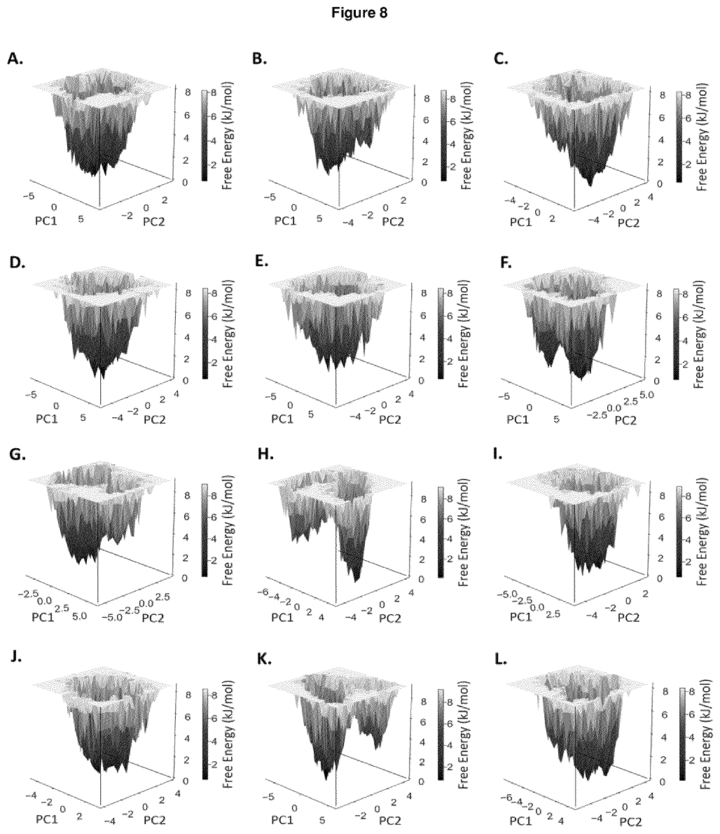

It is apparent from the figure that majority of the HLA-epitope complexes show a global collective dynamics except for HLA-DQA1*05:01/HLA-DQB1*03:01-epitope-2.2.1 (see FIG. 6H), HLA-DQA1*01:01/HLA-DQB1*05:01-2.2.4 (see FIG. 6K) and HLA-DQA1*05:01/HLA-DQB1*03:01-epitope-2.2.4 (see FIG. 6L). The widespread conformational subspace indicates that the HLA molecules manoeuvre through a broad conformational space before achieving an equilibrated state. As stated earlier, in some cases (DQA1*05:01/HLA-DQB1*03:01-epitope-2.2.1 and DQA1*05:01/HLA-DQB1*03:01-epitope-2.2.4), a smaller cluster of conformations is observed, which indicates higher flexibility of the HLA molecules, and is consistent with the RMSD plot (orange and blue lines in top-right panel of FIG. 3). To further understand the effect of epitope binding on the conformational redistribution and the energetics of the HLA molecules, free energy landscapes (FEL) was determined as the function of the first two principal components, PC1 and PC2. The 2D and 3D FEL plots for the HLA-epitope complexes are shown in FIG. 7 and FIG. 8, respectively.

As can be seen from these figures, the FEL of the MHC-I molecules consists of a broad minima with multiple conical ends suggesting a widespread distribution of low-energy conformations. Similar is the case with three MHC-II molecules, i.e., HLADQA1*04:01/HLADQB1*04:02 (FIG. 8G), HLA-DRB1*09:01 (FIG. 81) and HLA-DPA1*01:03/HLA-DPB1*02:01 (FIG. 8J). However, in case of other MHC-II molecules, such as HLA-DQA1*05:01/HLA-DQB1*03:01 (FIG. 8H) and HLA-DQA1*01:01/HLA-DQB1*05:01 (FIG. 8K), we observe multiple minima separated by small energy barriers in a broad basin, which indicates that epitope binding induces selection of multiple conformations of the HLA molecules, but only one minima consist of low-energy conformation. Thus, both the essential dynamics and FEL analysis indicate although different but stable binding stability of the HLA-epitope complexes.

While the world continues to suffer from the COVID-19 disease, the causal organism, i.e., SARS-CoV-2 that has jumped off or spilled over humans from an animal reservoir continues to evolve over time to incorporate mutations which increases its survival and infection rate. The COVID-19 outbreak, which was declared as pandemic by WHO within three months of the first report of the disease, perdures to remain, arguably, one of the biggest threat and mystery to mankind and also science, as still a lot of questions like, how the virus infects, spreads, survives, mutates, etc. remains unclear. However, given the collective efforts made by the scientific community across the world who have been working indefatigably to understand these questions to come up with preventive measures as well as therapeutic agents has led to the development of some RNA and vector-based vaccines, which have been approved under Emergency Used Authorization (EUA).

These approved vaccines from Moderna, Pfizer-BioNTech and Oxford-AstraZeneca have shown 62-95% efficacy in phase-3 or phase-2/3 clinical trials, however, they still possess several challenges including storage at ultra-low temperature, stability, scalability, high costs and allergic reactions. Synthetic vaccines based on peptides can minimize these challenges. Peptide-based vaccines have the ability to generate epitope-specific immune response and are more stable and easily accessible under normal storage conditions. Since peptide-based vaccines are chemically synthesised, the peptide-based vaccines can be manufactured at large scale with low cost. Unfortunately, none of the SARS-CoV-2 vaccines rolled-out to date in the market are peptide-based, as the majority of the focus of the scientific and industrial community has been on vector-based, whole pathogen, DNA, RNA and recombinant protein-based vaccines.

However, like any other vaccine development approaches, peptide vaccines also possess some limitations including reduced immunogenicity and enzymatic degradation. Such weakness could be improved by combining an adjuvant or particulate delivery carriers.

The present invention involves in silico design of B-cell and T-cell consensus epitopes (peptides) against SARS-CoV-2 by deploying various immunoinformatics tools and bioinformatics databases followed by confirming the efficacy of the designed epitopes against various HLA (MHC-I and II) proteins using molecular docking, molecular dynamics simulation and binding free energy estimation with MM-PBSA. Epitope prediction tools have been meticulously selected from a pool of software available in the immunoinformatics research literature based on their documented performance on the area under the receiver operating characteristics (ROC) curve (AUC) metric. Knowing the variability of results given by B-cell and T-cell epitope predictors, it was ensured to build consensus of results by deploying multiple methods and also applied a battery of immunological properties' tests to further refine results and maximize chances of identifying the best vaccine candidates.

SARS-CoV-2 spike glycoprotein was predicted to be antigenic as per VaxiJen at the start of the analysis, hence epitope candidates were expected to be identified throughout the protein. Nevertheless, it was necessary to refine the analysis to identify the best candidates which fit essential properties of a good peptide-based vaccine candidate, such as immunogenicity, allergenicity and population coverage, etc. The best four B-cell and T-cell epitope candidates were found in S1 regions of SARS-CoV-2 spike protein, which aligns with the fact the most of the recent epitope prediction results against SARS-CoV-2 have been focused on S1 region. However, the epitopes of the invention were concentrated around N-terminal domain (NTD) and C-terminal domain 2 (CTD2) rather than the Receptor Binding Domain (RBD), which interacts with the human ACE2 receptor to facilitate entry of SARS-CoV-2 in human target cells. In a recent study, it has been proposed that the flat sialic acid-binding domain at the N-terminal domain (NTD) of the S1 subunit plays a crucial role in fast motion over respiratory epithelium and ACE2 receptor scanning that allow SARS-CoV-2 rapid cellular entry. And also no high-frequency mutations have been detected so far in the C-terminal domain (CTD) of the S1 subunit. In light of the above and given the small sizes and immunogenic properties, the final four epitopes remain viable and better candidates for vaccine development when compared to the whole spike glycoprotein or the S1 or S2 subunits, which unfortunately in the case of SARS-CoV had shown potential to cause lung pathology.

SARS-CoV-2 virus is continuing to mutate and evolve, but fortunately none of the reported variants (https://www.cdc.gov/coronavirus/2019-ncov/transmission/variant.html) fall in the regions of our predicted epitopes.

The effectiveness of a vaccine from a public health program point of view depends on specificity and the level of population coverage among many other factors. Conservation analysis of the final four epitopes confirmed that they are unique and thus specific for SARS-CoV-2 found in humans as a host. Population coverage analysis confirmed that when the final four epitopes are used as a set they are able to cover 99.82% of the overall world population (see Table 4), indicating the development of a multi-epitope vaccine may provide better protection against the SARS-CoV-2 virus. Considering the technical constraints, expertise and infra-structure needed for developing multi-epitope vaccine, especially in low income countries, and thus from the health economics and value-for-money perspective, epitope number 2 with its broad allele specificity (see Table 3) would be a useful vaccine candidate. Epitope number 2 can cover 98% of the overall world population on its own (see Table 4) and at the same time is very unique to SARS-CoV-2 isolated from human as a host species (see Table 5).

Interestingly, all the final four epitopes showed less population coverage ranging between 0.00%-59.15% in the South African population (Table 2), which has also been previously demonstrated for other vaccines. Furthermore, SARS-CoV-2 vaccines from Moderna, Pfizer-BioNTech and Oxford-AstraZeneca have also been found to be less efficacious against South African population. With the recurrent evidence of poor efficacy of vaccines against South African population, it has become inevitably important that more systematic and focused approaches are needed for vaccine development for South African population.

The above results were further validated with molecular docking and MD simulation experiments. Complemented with MM-PBSA calculations, essential dynamics analysis and free energy landscape analysis, the results indicate a remarkable binding affinity of the five constituent t-cell epitopes of consensus epitope number 2 towards their corresponding HLA (MHC-I and II) proteins. The van der Waals interactions appeared to be the dominant factor responsible for the stability of the HLA-epitope complexes.

The present invention provides a profound biophysical insight into the factors and energetics stabilizing the HLA-epitope complexes of the five constituent t-cell epitopes of consensus epitope number 2, which is needed for triggering epitope-specific immune response against SARS-CoV-2. The current global pandemic due to SARS-CoV-2 has taken a substantial number of lives across the world. Although some vaccines have been rolled-out, a number of vaccine candidates are still under clinical trial at various pharmaceutical companies and laboratories around the world. Considering the variable nature of the virus which is continuing to mutate and evolve, persistent efforts are needed to develop better vaccine candidates. In the present invention, various immunoinformatics tools and bioinformatics databases were deployed to derive consensus B-cell and T-cell epitope sequences of SARS-CoV-2 spike glycoprotein. This approach has identified four potential epitopes which have the capability to initiate both antibody and cell mediated immune responses, are non-allergenic and do not trigger autoimmunity. These peptide sequences were also evaluated to show 99.82% of global population coverage based on the genotypic frequencies of HLA binding alleles for both MHC class-I and class-Il and are unique for SARS-CoV-2 isolated from human as a host species. Epitope number 2 alone had a global population coverage of 98.2%. Therefore, binding and interaction of the constituent T-cell epitopes with their corresponding HLA proteins was further validated using molecular docking and molecular dynamics simulation experiments, followed by binding free energy calculations with MM-PBSA, essential dynamics analysis and free energy landscape analysis. Previously, efforts towards utilising epitope prediction for designing a peptide-based vaccine against SARS-CoV-2 are either focused on only T-cell epitopes or on HLA allele frequencies amongst specific populations such as Japan or China. Some have focused on virus E protein, whereas others have investigated homology between SARS-CoV and SARS-CoV-2 to derive both common and unique epitopes. In the present invention, various immunoinformatics tools and bioinformatics databases have been deployed to predict B-cell and T-cell consensus epitopes as peptide-based vaccine candidates for SARS-CoV-2, which show maximum population coverage across all continents and thus can be effective globally. Recognition of the antigenic epitope was carried out strategically in such a way that the selected epitopes are capable of generating both antibody and cell-mediated immune responses. The designed epitopes are also predicted to be non-allergenic and show no autoimmune response in humans. The binding of constituent T-cell epitopes of the best consensus epitope with the corresponding HLA proteins were computationally validated using molecular docking and molecular dynamics simulation experiments.

1. An isolated polypeptide having the amino acid sequence defined in any one of SEQ ID NO:2, SEQ ID NO:4, SEQ ID NO:3, and SEQ ID NO:1 or an amino acid sequence having at least 40% identity thereto, or a fragment each thereof.

2. The isolated polypeptide of claim 1, wherein the isolated polypeptide has an amino acid sequence having at least 90% identity to the amino acid sequence defined in any one of SEQ ID NO:2, SEQ ID NO:4, SEQ ID NO:3, and SEQ ID NO:1, or a fragment each thereof.

3. The isolated polypeptide of claim 1, wherein the isolated polypeptide has the amino acid sequence defined in SEQ ID NO:2 or an amino acid sequence having at least 40% identity thereto, or a fragment each thereof.

4. The isolated polypeptide of claim 1, wherein the fragment has an amino acid sequence having at least 40% identity to the amino acid sequence defined in any of SEQ ID NO:5-SEQ ID NO:19.

5. The isolated polypeptide of claim 1, wherein the fragment has an amino acid sequence having at least 40% identity to the amino acid sequence defined in either or both of SEQ ID NO:1 and SEQ ID NO:17.

6. The isolated polypeptide of claim 1, wherein the fragment has an amino acid sequence having at least 40% identity to the amino acid sequence defined in any of SEQ ID NO:20-SEQ ID NO:33.

7. The isolated polypeptide of claim 1, wherein the fragment has an amino acid sequence having at least 40% identity to the amino acid sequence defined in either or both of SEQ ID NO:20 and SEQ ID NO:28.

8. The isolated polypeptide of claim 1, wherein the fragment has an amino acid sequence having at least 40% identity to the amino acid sequence defined in any of SEQ ID NO:34-SEQ ID NO:39.

9. The isolated polypeptide of claim 1, wherein the fragment has an amino acid sequence having at least 40% identity to the amino acid sequence defined in either or both of SEQ ID NO:34 and SEQ ID NO:37.

10. The isolated polypeptide of claim 1, wherein the fragment has an amino acid sequence having at least 40% identity to the amino acid sequence defined in any of SEQ ID NO:41-SEQ ID NO:55.

11. The isolated polypeptide of claim 1, wherein the fragment has an amino acid sequence having at least 40% identity to the amino acid sequence defined in either or both of SEQ ID NO:41 and SEQ ID NO:50.

12. (canceled)

13. A vaccine composition comprising an isolated polypeptide according to claim 1.

14. (canceled)

15. (canceled)

16. A method for generating an immune response in a subject comprising administering the isolated polypeptide according to claim 1 to a subject.

17. The method of claim 16, wherein the immune response is a humoral and cell-mediated immune response.

18. A method for vaccinating a subject comprising administering the isolated polypeptide according to claim 1 to a subject.

19. The method of claim 18, wherein the subject is vaccinated against a viral infection.

20. The method of claim 19, wherein the viral infection is severe acute respiratory syndrome coronavirus 2 infection.
